(19) United States
(12) Patent Application Publication
(10) Pub. No.: US 2024/0042028 A1
(43) Pub. Date: Feb. 8, 2024

(54) METHODS OF NK CELL ENGINEERING TO ENHANCE TUMOR TARGETING

(71) Applicant: The Johns Hopkins University, Baltimore, MD (US)

(72) Inventors: Challice Bonifant, Baltimore, MD (US); Ilias Christodoulou, Baltimore, MD (US)

(21) Appl. No.: 18/256,326

(22) PCT Filed: Dec. 7, 2021
(86) PCT No.: PCT/US2021/062251

§ 371 Date: Jun. 7, 2023

Related U.S. Application Data
(60) Provisional application No. 63/122,442, filed on Dec. 7, 2020.

(51) Int. Cl. A61K 39/00; C07K 14/725; C07K 16/28; A61P 35/02

(52) U.S. Cl. CPC A61K 39/4631; C07K 14/7051; C07K 16/2866; A61K 39/4611; A61K 39/4613; A61K 39/4643; A61P 35/02; C07K 2319/03; C07K 2317/53; C07K 2319/30

(57) ABSTRACT

Provided herein are, inter alia, methods, compositions and kits for treating cancer, e.g., acute myeloid leukemia, including an engineered cell including various CAR constructs. Also included herein are kits for treating cancer, including engineered cells comprising various CAR constructs.

The present application claims the benefit of priority under 35 U.S.C. 119 from U.S. provisional application No. 63/122,442, filed Dec. 7, 2020, which is incorporated by reference herein in its entirety.

### BACKGROUND

New compositions and methods for treating cancer, e.g., acute myeloid leukemia.

### BRIEF SUMMARY

Provided herein are, inter alia, methods, compositions and kits for treating and preventing cancer, e.g., acute myeloid leukemia. Also included herein are kits for treating cancer, e.g., acute myeloid leukemia.

In aspects, provided herein is an engineered cell including a chimeric antigen receptor (CAR) polypeptide comprising a hinge domain, a transmembrane domain, and/or an intracellular domain. In embodiments, the cell comprises an immune cell, wherein the immune cell comprises natural killer (NK) cells or T cells. In embodiments, the immune cell comprises NK cells.

In embodiments, the engineered cell further comprises a signaling domain, an activating domain, a stimulatory domain, an antigen recognition domain, or a co-stimulatory domain. For example, the signaling domain includes an immunoreceptor tyrosine-based activation motif (ITAM).

In aspects, the engineered cell comprises a hinge domain. For example, the hinge domain includes 2B4 comprising the sequence QDCQNAHQEFRFWP (SEQ ID NO: 1), FcεR1γ comprising the sequence LGEPQ (SEQ ID NO: 2), or DAP10 comprising the sequence QTTPGERSSLPAFYPGTSGSCSGCGSLSLP (SEQ ID NO: 3). In other examples, the hinge domain includes CD8α, including the sequence: TTTPAPRPPTPAPTIASQPLSLRPEACRPAAGGAVHTRGLDFACD (SEQ ID NO: 16) or CD8β, including the sequence: DFLPTTAQPTKKSTLKKRVCRLPRPETQKGPLCSP (SEQ ID NO: 17) or IgG4, including the sequence: EPKSCDKTHTCPPCPD (SEQ ID NO: 21).

In aspects, the engineered cell comprises a transmembrane region. The transmembrane region, includes for example, 2B4, having the sequence FLVIIVILSALFLGTLACFCV (SEQ ID NO: 29), FcεR1γ having the sequence LCYILDAILFLYGIVLTLLYC (SEQ ID NO: 30), or DAP10 having the sequence LLAGLVAADAVASLLIVGAVF (SEQ ID NO: 31). In other examples, the transmembrane region includes NKG2D, having the sequence PFFFCCFIAVAMGIRFIIMVA (SEQ ID NO: 18), CD8α having the sequence IYIWAPLAGTCGVLLLSLVIT (SEQ ID NO: 19), or CD8β having the following sequence ITLGLLVAGVLVLLVSLGVAI (SEQ ID NO: 20).

In aspects, the engineered cell includes an intracellular domain. The intracellular domain includes:

2B4:
(SEQ ID NO: 4)
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYS,
CD3ζ (1XX):
(SEQ ID NO: 5)
RVKFSRSADAPAYQQGQNQLYNELNLGRREEYDVLDKRRGRDPEMGGKPQ
RRKNPQEGLFNELQKDKMAEAFSEIGMKGERRRGKGHDGLFQGLSTATKD
TFDALHMQALPPR,
CD3ζ:
(SEQ ID NO: 6)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR,
CD3ζ (1Δ):
(SEQ ID NO: 7)
RVKFSRSADAPAYQQGQNQLYNELNLGRREEYDVLDKRRGRDPE,
FcϵR1γ
(SEQ ID NO: 8)
RLKIQVRKAAITSYEKSDGVYTGLSTRNQETYETLKHEKPPQ,
or
DAP10:
(SEQ ID NO: 9)
LCARPRRSPAQEDGKVYINMPGRG.

In aspects, the engineered cell described herein further includes an internal ribosome entry site (IRES) domain. For example, the nucleotide sequence of the IRES domain has the sequence:

(SEQ ID NO: 12)
CCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCT
TTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCT
CTGCGGCCAAAAGCCACGTGTATAAGATACACCTGCAAAGGCGGCACAAC
CCCAGTGCCACGTTGTGAGTTGGATAGTTGTGGAAAGAGTCAAATGGCTC
TCCTCAAGCGTATTCAACAAGGGGCTGAAGGATGCCCAGAAGGTACCCCA
TTGTATGGGATCTGATCTGGGGCCTCGGTGCACATGCTTTACATGTGTTT
AGTCGAGGTTAAAAAACGTCTAGGCCCCCCGAACCACGGGGACGTGGTTT
TCCTTTGAAAAACACGATGATAATATGGCCACAACC

In further aspects, the engineered cell described herein further includes a cytokine. For example, the cytokine includes interleukin-15 (IL-15), interleukin 21 (IL-21), interleukin 18 (IL-18), interleukin 12 (IL-12), or interleukin 2 (IL-2). The cytokine comprises the mature sequence, e.g., without the leader sequence.

For example, the IL-15 has the sequence:

(SEQ ID NO: 10)
MATTRISKPHLRSISIQCYLCLLLNSHFLTEAGIHVFILGCFSAGLPKTE
ANWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTS

For example, the IL-21 has the sequence:

(SEQ ID NO: 11)
MRSSPGNMERIVICLMVIFLGTLVHKSSSQGQDRHMIRMRQLIDIVDQLK
NYVNDLVPEFLPAPEDVETNCEWSAFSCFQKAQLKSANTGNNERIINVSI
KKLKRKPPSTNAGRRQKHRLTCPSCDSYEKKPPKEFLERFKSLLQKMIHQ
HLSSRTHGSEDS

For example, the IL-18 has the sequence:

(SEQ ID NO: 13)
MAAEPVEDNCINFVAMKFIDNTLYFIAEDDENLESDYFGKLESKLSVIRN
LNDQVLFIDQGNRPLFEDMTDSDCRDNAPRTIFIISMYKDSQPRGMAVTI
SVKCEKISTLSCENKIISFKEMNPPDNIKDTKSDIIFFQRSVPGHDNKMQ
FESSSYEGYFLACEKERDLFKLILKKEDELGDRSIMFTVQNED

In examples, the IL-12 has the sequence:

(SEQ ID NO: 14)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS

In further examples, the IL-2 has the sequence:

(SEQ ID NO: 15)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

In aspects, the engineered cell described herein provides that a cytokine is released (or secreted). For example, IL-15 or IL-21 is secreted. In other examples, IL-18, IL-12 or IL-2 are released or secreted.

In aspects, the engineered cell described herein includes the following structures:
2B4.z(truncated).IRES.secrIL15

The 2B4.z(truncated) sequence is provided below:

(SEQ ID NO: 22)
MDWIWRILFLVGAATGAHSQVQLQQPGAELVRPGASVKLSCKASGYTFTS
YWMNWVKQRPDQGLEWIGRIDPYDSETHYNQKFKDKAILTVDKSSSTAYM
QLSSLTSEDSAVYYCARGNWDDYWGQGTTLTVSSGGGGSGGGGSGGGGSD
VQITQSPSYLAASPGETITINCRASKSISKDLAWYQEKPGKTNKLLIYSG
STLQSGIPSRFSGSGSGTDFTLTISSLEPEDFAMYYCQQHNKYPYTFGGG
TKLEIKSGGGGSQDCQNAHQEFRFWPFLVIIVILSALFLGTLACFCVWRR
KRKEKQSETSPKEFLTIYEDVKDLKTRRNHEQEQTFPGGGSTIYSMIQSQ
SSAPTSQEPAYTLYSLIQPSRKSGSRKRNHSPSFNSTIYEVIGKSQPKAQ
NPARLSRKELENFDVYSGAGRVKFSRSADAPAYQQGQNQLYNELNLGRRE
EYDVLDKRRGRDPE

secrIL15 (secreted IL-15; SEQ ID NO: 10):

MATTRISKPHLRSISIQCYLCLLLNSHFLTEAGIHVFILGCFSAGLPKTE
ANWVNVISDLKKIEDLIQSMHIDATLYTESDVHPSCKVTAMKCFLLELQV
ISLESGDASIHDTVENLIILANNSLSSNGNVTESGCKECEELEEKNIKEF
LQSFVHIVQMFINTS

2B4.z(1XX).IRES.secIL15, where the 2B4.z(1XX) sequence is provided below:

(SEQ ID NO: 23)
MDWIWRILFLVGAATGAHSQVQLQQPGAELVRPGASVKLSCKASGYTFTS
YWMNWVKQRPDQGLEWIGRIDPYDSETHYNQKFKDKAILTVDKSSSTAYM
QLSSLTSEDSAVYYCARGNWDDYWGQGTTLTVSSGGGGSGGGGSGGGGSD
VQITQSPSYLAASPGETITINCRASKSISKDLAWYQEKPGKTNKLLIYSG
STLQSGIPSRFSGSGSGTDFTLTISSLEPEDFAMYYCQQHNKYPYTFGGG
TKLEIKSGGGGSQDCQNAHQEFRFWPFLVIIVILSALFLGTLACFCVWRR
KRKEKQSETSPKEFLTIYEDVKDLKTRRNHEQEQTFPGGGSTIYSMIQSQ
SSAPTSQEPAYTLYSLIQPSRKSGSRKRNHSPSFNSTIYEVIGKSQPKAQ
NPARLSRKELENFDVYSGAGRVKFSRSADAPAYQQGQNQLYNELNLGRRE
EYDVLDKRRGRDPEMGGKPQRRKNPQEGLFNELQKDKMAEAFSEIGMKGE
RRRGKGHDGLFQGLSTATKDTFDALHMQALPPR

2B4.z.IRES.secIL15, where the 2B4.z sequence is provided below:

(SEQ ID NO: 24)
MDWIWRILFLVGAATGAHSQVQLQQPGAELVRPGASVKLSCKASGYTFTS
YWMNWVKQRPDQGLEWIGRIDPYDSETHYNQKFKDKAILTVDKSSSTAYM
QLSSLTSEDSAVYYCARGNWDDYWGQGTTLTVSSGGGGSGGGGSGGGGSD
VQITQSPSYLAASPGETITINCRASKSISKDLAWYQEKPGKTNKLLIYSG
STLQSGIPSRFSGSGSGTDFTLTISSLEPEDFAMYYCQQHNKYPYTFGGG
TKLEIKSGGGGSQDCQNAHQEFRFWPFLVIIVILSALFLGTLACFCVWRR
KRKEKQSETSPKEFLTIYEDVKDLKTRRNHEQEQTFPGGGSTIYSMIQSQ
SSAPTSQEPAYTLYSLIQPSRKSGSRKRNHSPSFNSTIYEVIGKSQPKAQ
NPARLSRKELENFDVYSGAGRVKFSRSADAPAYQQGQNQLYNELNLGRRE
EYDVLDKRRGRDPEMGGKPQRRKNPQEGLYNELQKDKMAEAYSEIGMKGE
RRRGKGHDGLYQGLSTATKDTYDALHMQALPPR

41BB sequence is provided below:

(SEQ ID NO: 25)
KRGRKKLLYIFKQPFMRPVQTTQEEDGCSCRFPEEEEGGCEL

In other aspects, the following CAR constructs are contemplated:
2B4.z(truncated).IRES.secrIL21 (2B4.z(truncated) sequence (SEQ ID NO: 22) and SEQ ID NO: 11), or
2B4.z(1XX).IRES.secIL21 (2B4.z(1XX) sequence (SEQ ID NO: 23) and SEQ ID NO: 11), or
2B4.z.IRES.secIL21 (2B4.z sequence (SEQ ID NO: 24) and SEQ ID NO: 11), or
2B4.z(truncated).IRES.secrIL18, (2B4.z(truncated) (SEQ ID NO: 22) and SEQ ID NO: 13), or
2B4.z(1XX).IRES.secIL18, (2B4.z(1XX) (SEQ ID NO: 23) and SEQ ID NO: 13), or
2B4.z.IRES.secIL18 (2B4.z sequence (SEQ ID NO: 24) and SEQ ID NO: 13), or
2B4.z(truncated).IRES.secrIL12, (2B4.z(truncated) sequence (SEQ ID NO: 22) and SEQ ID NO: 14), or
2B4.z(1XX).IRES.secIL12 (2B4.z(1XX) sequence (SEQ ID NO: 23) and SEQ ID NO: 14), or
2B4.z.IRES.secIL12 (2B4.z sequence (SEQ ID NO: 24) and SEQ ID NO: 14).

In embodiments, provided herein is a method for treating a cancer, e.g., acute myeloid leukemia (AML), where the method includes administering to a subject in need thereof an effective of the engineered cells disclosed herein including a chimeric antigen receptor (CAR) polypeptide comprising a hinge domain, a transmembrane domain, and/or an intracellular domain. In embodiments, the cell comprises an immune cell, wherein the immune cell comprises natural killer (NK) cells or T cells. For example, the immune cell includes NK cells. Suitable subjects include mammals such as humans, in particular a human that has been diagnosed with cancer, such as acute myeloid leukemia (AML).

Pharmaceutical compositions and treatment kits are also provided that comprise engineered cells as disclosed herein.

As described herein, the composition comprising the engineered cell including a CAR has significant advantages compared to current therapies.

As further discussed below, in specific embodiments, NK cell populations were characterized expressing a panel of AML (CD123)-specific CARs and/or IL15 in vitro and in AML xenograft models. The CD123-specific scFV (26292) sequence is provided below:

(SEQ ID NO: 26)
QVQLQQPGAELVRPGASVKLSCKASGYTFTSYWMNWVKQRPDQGLEWIGR
IDPYDSETHYNQKFKDKAILTVDKSSSTAYMQLSSLTSEDSAVYYCARGN
WDDYWGQGTTLTVSSGGGGSGGGGSGGGGSDVQITQSPSYLAASPGETIT
INCRASKSISKDLAWYQEKPGKTNKLLIYSGSTLQSGIPSRFSGSGSGTD
FTLTISSLEPEDFAMYYCQQHNKYPYTFGGGTKLEIK.

scFv (26292).2B4.z.IRES.sIL15 sequence is provided below:

(SEQ ID NO: 27)
atggactggatctggcgcatcctgtttctcgtgggagccgccacaggcgc
ccattctcaggtgcagctgcagcagcctggcgctgaactcgtgcggccag
gcgcttctgtgaagctgagctgtaaagccagcggctacaccttcaccagc
tactggatgaactgggtcaagcagcggcccgaccagggcctggaatggat
cggaagaatcgacccctacgacagcgagacacactacaaccagaagttca
aggacaaggccatcctgaccgtggacaagagcagcagcaccgcctacatg
cagctgtccagcctgaccagcgaggacagcgccgtgtactactgcgccag
aggcaactgggacgactactggggccagggcacaaccctgacagtgtcta
gcggaggcggaggaagtggcggcggaggatctgggggaggcggatctgat
gtgcagatcacccagagccccagctacctggctgcctctcctggcgagac
aatcaccatcaactgccgggccagcaagagcatctccaaggacctggcct
ggtatcaggaaaagcccggcaagaccaacaagctgctgatctacagcggc
agcaccctgcagagcggcatccccagcagattttccggcagcggctccgg
caccgacttcaccctgacaatcagctccctggaacccgaggactttgcca
tgtactattgccagcagcacaacaagtacccttacaccttcggcggaggc
accaagctggaaatcaagagcggagggggcggatcccaggattgccagaa
tgcccaccaagagttccggttctggcccttcctggtcatcatcgtgatcc
tgagcgccctgttcctgggcaccctggcctgtttttgcgtgtggcgcaga
aagcgcaaagagaagcagagcgagacaagccccaaagagttcctgaccat
ctacgaggacgtgaaggacctgaaaacccggcggaaccacgagcaagagc
agacctttcctggcggcggaagcaccatctacagcatgatccagagccag
agcagcgcccctacaagccaagagcctgcctacacactgtactccctgat
ccagcctagcagaaagagcggcagccggaagagaaatcacagccccagct
tcaacagcacgatctacgaagtgatcggcaagagccagccaaaggctcag
aaccctgccagactgagccggaaagagctggaaaacttcgacgtgtactc
tggggccggcagagtgaagttcagcagatcagccgatgctcccgcctatc
agcagggccagaaccagctgtacaacgagctgaacctggggagaagagaa
gagtacgacgtgctggacaagcggagaggcagagatcctgagatgggcgg
aaagccccagcggagaaagaatcctcaagagggcctgtataatgagctgc
agaaagacaagatggccgaggcctacagcgagatcggaatgaagggcgag
cgcagaagaggcaagggacacgatggactgtaccagggcctgagcaccgc
caccaaggatacctatgatgccctgcacatgcaggccctgcctccaagat
gagcggccgccgcatgctacgtaaattccgcccctctccctccccccccc
ctaacgttactggccgaagccgcttggaataaggccggtgtgcgtttgtc
tatatgttattttccaccatattgccgtcttttggcaatgtgagggcccg
gaaacctggccctgtcttcttgacgagcattcctaggggtctttcccctc
tcgccaaaggaatgcaaggtctgttgaatgtcgtgaaggaagcagttcct
ctggaagcttcttgaagacaaacaacgtctgtagcgaccctttgcaggca
gcggaaccccccacctggcgacaggtgcctctgcggccaaaagccacgtg
tataagatacacctgcaaaggcggcacaaccccagtgccacgttgtgagt
tggatagttgtggaaagagtcaaatggctctcctcaagcgtattcaacaa
ggggctgaaggatgcccagaaggtaccccattgtatgggatctgatctgg
ggcctcggtgcacatgctttacatgtgtttagtcgaggttaaaaaacgtc
taggccccccgaaccacggggacgtggttttcctttgaaaaacacgatga
taatatggccacaaccaggatcagcaagccccacctgagatccatcagca
tccagtgctacctgtgcctgctgctgaacagccacttcctgacagaggcc
ggaatccatgtgttcatcctgggctgctttagcgccggactgcctaagac
cgaagccaactgggtcaacgtgatcagcgacctgaagaagatcgaggacc
tgatccagagcatgcacatcgacgccacactgtacaccgagtccgatgtg
caccccagctgtaaagtgaccgccatgaagtgctttctgctggaactgca
agtgatcagcctggaaagcggcgacgccagcatccacgacaccgtggaaa
acctgatcatcctggccaacaacagcctgagcagcaacggcaatgtgacc
gagagcggctgcaaagagtgcgaggaactggaagagaagaatatcaaaga
gttcctgcagagcttcgtccacatcgtgcagatgttcatcaacaccagct
ga.

sIL15.IRES.mO is provided below:

(SEQ ID NO: 28)
atgaggatcagcaagccccacctgagatccatcagcatccagtgctacct
gtgcctgctgctgaacagccacttcctgacagaggccggaatccatgtgt
tcatcctgggctgctttagcgccggactgcctaagaccgaagccaactgg
gtcaacgtgatcagcgacctgaagaagatcgaggacctgatccagagcat
gcacatcgacgccacactgtacaccgagtccgatgtgcaccccagctgta
aagtgaccgccatgaagtgctttctgctggaactgcaagtgatcagcctg
gaaagcggcgacgccagcatccacgacaccgtggaaaacctgatcatcct
ggccaacaacagcctgagcagcaacggcaatgtgaccgagagcggctgca
aagagtgcgaggaactggaagagaagaatatcaaagagttcctgcagagc
ttcgtccacatcgtgcagatgttcatcaacaccagctgagcggccgccgc
atgctacgtaaattccgcccctctccctcccccccccctaacgttactgg
ccgaagccgcttggaataaggccggtgtgcgtttgtctatatgttatttt
ccaccatattgccgtcttttggcaatgtgagggcccggaaacctggccct
gtcttcttgacgagcattcctaggggtctttcccctctcgccaaaggaat
gcaaggtctgttgaatgtcgtgaaggaagcagttcctctggaagcttctt
gaagacaaacaacgtctgtagcgaccctttgcaggcagcggaacccccca
cctggcgacaggtgcctctgcggccaaaagccacgtgtataagatacacc
tgcaaaggcggcacaaccccagtgccacgttgtgagttggatagttgtgg
aaagagtcaaatggctctcctcaagcgtattcaacaaggggctgaaggat
gcccagaaggtaccccattgtatgggatctgatctggggcctcggtgcac
atgctttacatgtgtttagtcgaggttaaaaaacgtctaggccccccgaa
ccacggggacgtggttttcctttgaaaaacacgatgataatatggccaca
agttagcaagggtgaggagaataacatggcgatcattaaggaatttatgc
gctttaaggtccggatggagggatcagtgaatggtcacgagttcgagatc
gagggcgagggagaaggaagaccttacgagggcttccagaccgcaaaact
gaaagttactaaaggggcccactccccttcgcctgggatattctcagtcc
gcaattcacatacggctctaaggcgtatgtaaaacaccctgccgacatcc
ctgactacttcaagctcagctttcccgagggctttaagtgggaacgggtc
atgaattttgaagatggaggagtcgttacagtgacccaagattcttcatt
gcaggatggcgagttcatttataaggtgaagctcaggggcaccaacttcc
cctcagatgggccagtgatgcagaaaaagactatgggctgggaagctagc
tccgagcgcatgtacccagaggatggcgcgctcaaaggggagattaagat
gagactgaaactgaaggacggaggacactatactagtgaggtaaagacta
cgtacaaagcaaagaaacccgtccagcttcctggagcgtacatcgtgggc
attaaactcgatattacttcacacaacgaggactacaccatcgtggagca
atatgagcgagccgagggtagacacagtacaggcggaatggacgagcttt
acaagtga.

In specific embodiments, CARs with 2B4.ζ or 4-1BB.ζ signaling domains demonstrated greater cell surface expression and endowed NK cells with improved anti-AML activity in vitro. As set forth in the examples which follow, initial in vivo testing revealed that only 2B4.ζ CAR-NK cells had improved anti-AML activity in comparison to untransduced (UTD) and 4-1BB.ζ CAR-NK cells. However, the benefit was transient due to limited CAR-NK cell persistence. Transgenic expression of secretory (s)IL15 in 2B4.ζ CAR and UTD NK cells improved their effector function in the setting of chronic antigen simulation in vitro. Multiparameter flow analysis after chronic antigen exposure identified the expansion of unique NK cell subsets. 2B4.ζ/sIL15 CAR and sIL15 NK cells maintained an overall activated NK cell phenotype. This was confirmed by transcriptomic analysis, which revealed a highly proliferative and activated signature in these NK cell groups. In vivo, 2B4.ζ/sIL15 CAR-NK cells had potent anti-AML activity in one model, while 2B4.ζ/sIL15 CAR and sIL15 NK cells induced lethal toxicity in a second model.

In certain aspects, an isolated natural killer (NK) cell comprises a chimeric antigen receptor (CAR) polypeptide comprising an antigen specific binding domain, a hinge domain, a transmembrane domain, an intracellular domain, a co-stimulatory receptor or combinations thereof, wherein the antigen specific binding domain specifically binds to CD123. In certain embodiments, the chimeric antigen receptor (CAR) polypeptide comprises a signaling domain that comprises an immunoreceptor tyrosine-based activation motif (ITAM). In certain embodiments, the transmembrane (TM) domain comprises 2B4, CD8α, FcεR1γ or DAP10. In certain embodiments, the co-stimulatory receptor comprises 2B4 and a T cell receptor CD3ζ chain. In certain embodiments, the co-stimulatory receptor comprises 4-1BB and a T cell receptor CD3ζ chain (4-1BB.ζ). In certain embodiments, the CD3ζ chain is truncated. In certain embodiments, the CD3ζ chain comprises one or more mutations. In certain embodiments, the CD3ζ chain comprises mutations of tyrosines at positions Y60, Y72, Y91, Y102 or combinations thereof. In certain embodiments, the tyrosines are substuted with phenylalanine. In certain embodiments, the CD123 specific binding domain comprises an antibody, antibody fragment or aptamer. In certain embodiments, the antibody fragment is a single chain fragment. In certain embodiments, the single chain fragment is a single chain variable fragment (scFv). In certain embodiments, the NK cell further comprises a cytokine. In certain embodiments, the cytokine comprises interleukin-15 (IL-15), interleukin-21 (IL-21), interleukin-18 (IL-18), interleukin-12 (IL-12), or interleukin-2 (IL-2).

In another aspect, a chimeric antigen receptor (CAR) comprises an antigen specific binding domain, a hinge domain, a transmembrane domain, and/or an intracellular domain. In certain embodiments, the CAR further comprises a signaling domain, an activating domain, a stimulatory domain, a co-stimulatory domain or combinations thereof. In certain embodiments, the chimeric antigen receptor (CAR) polypeptide comprises a signaling domain that comprises an immunoreceptor tyrosine-based activation motif (ITAM). In certain embodiments, the hinge domain comprises 2B4 comprising the sequence QDCQNAHQEFRFWP (SEQ ID NO: 1), FcεR1γ comprising the sequence LGEPQ (SEQ ID NO: 2), or DAP10 comprising the sequence QTTPGERSSLPAFYPGTSGSCSGCGSLSLP (SEQ ID NO: 3). In certain embodiments, the transmembrane region comprises 2B4, comprising the sequence FLVIIVILSALFLGTLACFCV (SEQ ID NO: 29), FcεR1γ comprising the sequence LCYILDAILFLYGIVLTLLYC (SEQ ID NO: 30), or DAP10 comprising the sequence LLAGLVAADAVASLLIVGAVF (SEQ ID NO: 31). In certain embodiments, the intracellular domain comprises 2B4 (SEQ ID NO: 4), CD3ζ (1XX) (SEQ ID NO: 5), CD3ζ (SEQ ID NO: 6), CD3ζ (1Δ), (SEQ ID NO: 7) FcεR1γ (SEQ ID NO: 8), or DAP10 (SEQ ID NO: 9). In certain embodiments, the chimeric antigen receptor further comprises a cytokine. In certain embodiments, the cytokine comprises interleukin-15 (IL-15), interleukin-21 (IL-21), interleukin-18 (IL-18), interleukin-12 (IL-12), or interleukin-2 (IL-2).

In another aspect, a chimeric antigen receptor (CAR) comprises an antigen specific binding domain, a hinge domain, a transmembrane domain, an intracellular domain, a co-stimulatory receptor or combinations thereof, wherein the antigen specific binding domain specifically binds to CD123. In certain embodiments, the chimeric antigen receptor (CAR) polypeptide comprises a signaling domain that comprises an immunoreceptor tyrosine-based activation motif (ITAM). In certain embodiments, wherein the transmembrane (TM) domain comprises 2B4, CD8α, FcεR1γ or DAP10. In certain embodiments, wherein the co-stimulatory receptor comprises 2B4 and a T cell receptor CD3ζ chain. In certain embodiments, the co-stimulatory receptor comprises 4-1BB and a T cell receptor CD3ζ chain (4-1BB.ζ). In certain embodiments, the CD3ζ chain is truncated. In certain embodiments, the CD3ζ chain comprises one or more mutations. In certain embodiments, the CD3ζ chain comprises mutations of tyrosines at positions Y60, Y72, Y91, Y102 or combinations thereof. In certain embodiments, the chimeric antigen receptor further comprises a cytokine. In certain embodiments, the cytokine comprises interleukin-15 (IL-15), interleukin-21 (IL-21), interleukin-18 (IL-18), interleukin-12 (IL-12), or interleukin-2 (IL-2). In certain embodiments, the CD123 specific binding domain comprises an antibody, antibody fragment or aptamer. In certain embodiments, the antibody fragment is a single chain fragment. In certain embodiments, the single chain fragment is a single chain variable fragment (scFv).

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C are data showing CAR Expression on NK cells FIG. 1A is a graph showing the percent CAR+NK cells measured by flow cytometry using recombinant CD123-His and anti-His-APC. Each spot is representative of a unique NK cell donor. FIG. 1B is a graph showing the mean fluorescence intensity of CAR+ cells, representative of CAR surface density. FIG. 1C are representative flow histogram plots for each CAR-NK cell population.

FIGS. 2A-2B are data showing CAR-NK cell anti-tumor activation and cytotoxicity. FIG. 2A is a bar graph showing the percent change in Interferon gamma (IFNγ) secretion by CAR-NK cells in co-culture with target negative (Raji) or target positive (MV-4-11) cell lines. FIG. 2B is a bar graph showing cytotoxicity of CAR-NK against target cells measured with flow cytometric counting following co-cultures at indicated effector:target (E:T) ratios.

FIGS. 3A-3D are data showing CAR-NK cell in vivo anti-tumor activity and persistence. FIG. 3A is a schematic of murine model. FIG. 3B is a graph showing NSG mice engrafted with MV-4-11.ffLuc (expressing firefly luciferase) treated with NK cells as indicated on D7. Leukemic progression monitored with weekly bioluminescence imaging. FIG. 3C is a graph showing the survival of mice. FIG. 3D is a graph showing peripheral blood queried for circulating human NK cells on D14, D21, and D28. Cells detected by flow cytometry following red blood cell lysis.

FIG. 4 are data showing enhanced short-term cytotoxicity with engineered IL15 secretion Co-culture assays performed with the indicated target cells stably engineered to express firefly luciferase. NK and CAR-NK cells were added at the indicated effector:target ratios and cells were cultured for 18-24 hours. Cytotoxicity was measured with detection of bioluminesce and correlation to identical conditions without effector cells present.

FIGS. 5A-5B are graphs showing that engineered Interleukin-15 (IL15) secretion improves CAR-NK persistence and cytotoxicity in a model of chronic antigen exposure.

FIG. 5A is a graph showing results of serial stimulation assays performed with addition of target cells (CD123+MV-4-11) at 1:1 effector:target ratio every 24 hours. NK cell counts measured with flow cytometric cell counting assay. FIG. 5B is a bar graph showing the cytotoxicity of NK cells engineered to express indicated CARs and/or secrete IL15 evaluated daily during serial stimulation assay by flow cytometry.

FIG. 6 is a bar graph showing that secreted Interleukin-15 (IL15) is detectable in the supernatant of cultured, engineered primary NK cells. Unmodified (UTD), and NK cells expressing the indicated CARs with and without engineered constitutive IL15 secretion were plated in cytokine-free media. After 24 hours, supernatant was collected and IL15 measured using ELISA.

FIGS. 7A-7C are data showing that mutation of distal CD3ζ ITAMs does not decrease CAR-NK functionality. FIG. 7A is a graph that shows the expression of 2B4.ζ(1XX) and 2B4.ζΔ CARs in primary NK cells with and without IL15 secretion. CAR expression measured with flow cytometry using recombinant CD123-His and anti-His-APC. FIG. 7B are graphs showing results of cytotoxicity of NK cells engineered to express 2B4.ζ(1XX) (black) or 2B4.ζ(1XX).IRES.sIL15 (red) in short term co-culture assay of engineered NK cells and the indicated target cells expressing ffLuc. Live cells measured using treatment with D-Luciferin and measurement of BLI. FIG. 7C is a bar graph showing that NK cell anti-leukemia cytotoxicity evaluated daily during serial stimulation assay with replacement of MV-4-11 to maintain a 1:1 effector:target ratio. NK cell and target cell numbers measured using flow cytometry.

FIG. 8 is a diagram showing NK cell alloreactivity versus AML.

FIGS. 9A and 9B are diagrams showing biologically-relevant CAR designs.

FIG. 10 is a diagram showing NK-cell expansion and modification for the clinical vision.

FIG. 11 is a bar graph showing that CD123-CAR NKs have specific activation.

FIG. 12 (includes FIGS. 12A-12D). NK cells engineered with anti-CD123 CARs have antigen-specific functionality. FIG. 12A: Schema of CAR design. All CARs bind CD123 via an extracellular single chain variable fragment (scFv). The hinge (H), transmembrane (TM) and intracellular (IC) domains of the CARs are as indicated. Colored boxes represents each particular CAR with colors carried through each figure. FIG. 12B: Percentage (%) of CAR (+) NK cells detected on day (D)8 and D18. FIG. 12C: Bar plot comparing the percentage of CAR (+) NK cells with indicated transmembrane (TM) domains on D8. FIG. 12D: Absolute number of target cells measured after 72 h co-culture with indicated NK cells. Initial target cell count was 100,000 in 1:1 and 250,000 in 1:5 E:T ratio conditions. Each bar representative of the mean plus or minus the standard error of mean (+/−SEM); each dot representative of individual NK cell donor. n=3 donors (*p<0.05; **p<0.01; ***p<0.001; ****p<0.0001).

FIG. 13 (includes FIGS. 13A-13E). Anti-CD123.2B4.ζ CAR-NKs have transient anti-AML activity in vivo. FIG. 13A: Schematic of MV-4-11 xenograft model. On day 0, NSG mice were injected via tail vein with 1×10⁶ CD123(+) MV-4-11 cells that express firefly Luciferase (MV-4-11.ffLuc cells). In treatment groups, 10×10⁶ NK cells were administered on day 7. Cohorts: untransduced/unmodified (UTD), 4-1BB.ζ CAR-NK, and 2B4.ζ CAR-NK. FIG. 13B: Leukemia proliferation was monitored with bioluminescence imaging and was recorded as photons/sec/cm²/sr; n=8-12 mice per group. Magnification of days 7-21 shown. FIG. 13C: Representative images of 3 mice per condition. Minimum and maximum values of color scale are depicted at top [min-max]. FIG. 13D: Kaplan-Meier survival analysis of MV-4-11 xenografts (n=8-12 mice per condition). FIG. 13E: Mouse peripheral blood (PB) collected at indicated time points and analyzed via flow cytometry. Each dot represents a single mouse. Solid line: median. At later time points, NK cell count was undetectable for all groups and is not plotted (*p<0.05; **p<0.01; ***p<0.001).

FIG. 14 (includes 14A-14G). Simultaneous 2B4.ζ CAR expression and IL15 secretion strengthens NK cell cytotoxicity. FIG. 14A: Schema of vectors and IL15 secretion from CAR-NK cells. FIG. 14B: Quantification of retroviral vector copy number (VCN) in transduced NK cells. UTD NK cells served as negative controls (n=4 donors). FIG. 14C: Percentage (%) of transduced NK cells in cultures on D8 (black circle) and D18 (white circle, n=4 donors). FIG. 14D: NK cell supernatant was used for quantification of IL15 by ELISA (n=7 biological replicates using 4 donors). BL based cytotoxicity assays performed using the CD123(+) AML cell lines MV-4-11(FIG. 14E) and MOLM-13 (FIG. 14F) with ffLuc expression (n=4 donors). Asterisks indicate 2B4.ζ/sIL15 vs 2B4.ζ comparison. (FIG. 14G: Bar graph comparing percent (%) cytotoxicity of NK cells against Raji (CD123(−): solid), and Raji.CD123 (CD123(+): diagonal stripe) cancer cell lines at 1:10 E:T ratio (n=4 donors). For panels B-G, mean+/−SEM represented (*p<0.05; **p<0.01; ***p<0.001, ****p<0.001).

FIG. 15 (includes FIGS. 15A-15E). IL15 maintains NK cell activated phenotype in a model of chronic antigen stimulation. FIG. 15A: Schematic representation of our serial stimulation assay. Day 0 was the day of the initial seeding of the co-culture; day 1 the first and day 10 (D10) the last day of cell quantification. Immunophenotypic analysis of effector and target cells performed at baseline (before co-culture), 12 hours after 1^{st} stimulation, and on D10 (n=1 donor). Transcriptomic analysis performed on D10 (n=3 donors). FIG. 15B: Heatmap of flow cytometry data showing expression of 15 different NK cell surface markers. Heatmap coloring represents arcsinh transformed median marker intensity. FIG. 15C: Bar plots of relative abundance of the 32 population subsets found in each sample. FIG. 15D: NK cell counts over a period of 10 days. Initial seeding count was 2 million NK cells (mean+/−SEM; n=3 donors). Asterisks indicate 2B4.ζ/sIL15 vs 2B4.ζ and sIL15 vs UTD comparison. FIG. 15E: Heat map of the percent (%) NK cell cytotoxicity. Each column represents the specific day and each row a unique biological replicate (n=3 donors).

FIG. 16 (includes FIGS. 16A-16E): IL15 stimulation of NK cells upregulates genes important to cell cycle progression, NK cell activation, and cytotoxicity. FIG. 16A: Principal component analysis (PCA) of NK cell transcriptome on D10 of the chronic antigen stimulation. Each dot represents a unique NK cell donor (n=3 donors). FIG. 16B: Volcano plots representing differentially regulated genes in 2B4.ζ/sIL15 compared to 2B4.ζ (orange) and sIL15 compared to unmodified cells (green). Grey dots are those not meeting criteria: p-value ≥0.05; fold change >2 or <½. FIG. 16C: Bar plot depicting the top 10 significantly different KEGG 2021 Human gene Set Enrichment pathways Dotted line at p-value of 0.05. FIG. 16D: Heatmap and hierarchical clustering performed on differentially expressed genes. Biologically relevant clusters and enriched pathways (shown on the bottom of the cluster) represented. Z-score scale bar at right. FIG. 16E: Dot plots displaying the normalized expression of genes encoding activating receptors (blue rectangle), inhibitory receptors/checkpoint molecules (red), proliferation/anti-apoptosis markers (green), inflammatory mediators (purple), death receptor ligands (grey) and activation (yellow) markers (n=3 independent donors; *p<0.05; **p<0.01; ***p<0.001; ****p<0.0001).

FIG. 17 (includes FIGS. 17A-17J): IL15 secreting CAR-NK cells can cause lethal toxicity in an AML xenograft model. FIG. 17A: Schematic of NK cell dosing in MV-4-11.ffLuc model. FIG. 17B: MV-4-11 proliferation was monitored with bioluminescence imaging (BLI). Representative images of mice. The minimum and maximum values of the color scale are indicated.[min-max]. FIG. 17C: BL representative of leukemia proliferation was recorded as photons/sec/cm²/sr. Dotted lines: individual mice, Solid lines: Mean (n=5-7 mice per group; 2 experiments performed for UTD and 2B4.ζ cohorts). FIG. 17D: Kaplan-Meier survival analysis. FIG. 17E: Mouse peripheral blood (PB) was collected at indicated time points and analyzed via flow cytometry. NK cell numbers per microliter of mouse PB were tracked starting on day 13 of the experiment. Each dot represents cell numbers from a single mouse; line is at median. Asterisks indicate 2B4.ζ/sIL15 vs 2B4.ζ comparison. FIG. 17F: Human IL15 from peripheral blood of MV-4-11 engrafted mice drawn at the indicated time points was quantified (pg/mL) with ELISA. Asterisks indicate 2B4.ζ/sIL15 vs 2B4.ζ comparison. FIG. 17G: Schematic of NK cell dosing in MOLM-13.ffLuc model. FIG. 17H: MOLM-13 proliferation was monitored using BLI (n=5 mice per group). Representative images, (FIG. 171) radiance, and (FIG. 17J) Kaplan-Meier survival analysis (*p<0.05; **p<0.01; ***p<0.001; ****p<0.0001).

FIG. 18 (includes FIGS. 18A-18C). CAR expression and proliferative rate of CAR-NK cells. FIG. 18A: FACS plots representing the gating strategy used to identify CAR+NK cells. FIG. 18B: Bar plot comparing the mean fluorescence intensities+/−SEM of CARs with indicated transmembrane (TM) domains. FIG. 18C: Rate of expansion of our CAR-NK cells calculated with manual counting of absolute number. Initial seeding count was 250,000 cells. Fold expansion indicated in [ ] to the right of legend. Mean+/−SEM (n=3 donors).

FIG. 19. Anti-CD123 CAR-NK cells are activated by CD123+ cells. Percent (%) change of IFNγ secretion from baseline measured at 24-hours in co-culture assays of indicated CAR-NKs with Raji [CD123(−)] and MV-4-11 [CD123 (+)] cancer cell lines. IFNγ secretion was measured with ELISA. Each bar representative of the mean plus or minus the standard error of mean (+/−SEM); each dot is representative of an individual NK cell donor.

FIG. 20 (includes FIGS. 20A-20B). NK and leukemia cell percentages in the bone marrow and spleen of MV-4-11 engrafted mice on experimental days 15 and 22. FIG. 20A: Gating strategy used to identify hCD45+ cells in all in vivo experiments. LiveDead negative cells (alive)→excluding cellular debris→Single cells→human CD45 positive (+) cells (human cells). FIG. 20B: FACS plots of hCD45(+) cells in the bone marrow and spleen of mice on day 15 (8 days after NK cell injection) and on day 22 (15 days after NK cell injection). MV-4-11.ffLuc cells (red rectangle) are GFP positive. Numbers represent percentages of NK or AML cells. One mouse analyzed per condition per day.

FIG. 21 (includes FIGS. 21A-21B): Expression of CARs and IL15 in NK cells. FIG. 21A: Mean fluorescence intensity (MFI) of CAR (2B4.ζ and 2B4.ζ/sIL15) or mOrange (sIL15/mO) expression on transduced NK cells (n=4 donors measured at 2 time points).

FIG. 21B: Transgenic IL15 expression measured with Real-Time quantitative PCR. Normalized gene expression plotted relative to donor #3 sIL15 condition. GAPDH used as reference gene for normalization of gene expression (n=3 donors).

FIG. 22 (includes FIGS. 22A and 22B). Surface antigen quantification on target cells. Quantification of (FIG. 22A) CD123 and (FIG. 22B) IL15Ra per cell (*p<0.05; ***p<0.001; ****p<0.0001).

FIG. 23 (includes FIGS. 23A and 23B): IL15-secreting CAR-NK cells demonstrate a distinct phenotype after chronic antigen stimulation. FIG. 23: Heatmap of flow cytometry data showing expression of 17 different NK cell surface markers. Heatmap coloring represents arcsinh transformed median marker intensities. FIG. 23B: Bar plots of relative abundance of the 31 population subsets found in each sample. Unstimulated, freshly isolated NK cells are used as controls.

FIG. 24: Distribution of marker intensities of indicated “Panel A” receptors in the identified 32 NK cell clusters. The NK cell clusters (on the left side) are named as elsewhere. Histograms represent the respective marker in each cluster. Reference (blue, top each panel) histograms are calculated from all cells.

FIG. 25 (includes FIGS. 25A-25C): Visual representation of “Panel A” immunophenotype data. FIG. 25A: Multidimensional scaling (MDS) plot. Unstimulated, freshly isolated NK cells are used as controls. Arrows indicate the transitions across timepoints of experiment. FIG. 25B: Uniform Manifold Approximation and Projection (UMAP) plot was generated based on the arcsinh-transformed expression of the 15 expression markers in the NK cells from the whole dataset. Cells are colored according to the 32 clusters generated after manually merging the 40 meta-clusters obtained with FlowSOM. FIG. 25C: Individual UMAP plots of different NK cell conditions. Different time points indicated on the left side (baseline, 12 h, D10).

FIG. 26: Distribution of marker intensities of the indicated “Panel B” receptors in the 31 NK cell clusters. The NK cell clusters (on the left side) are specific for FIG. 22. Histograms represent the respective marker in each cluster. Reference (blue, top each panel) histograms are calculated from all cells.

FIG. 27 (includes FIGS. 27A-27C). Visual representation of “Panel B” immunophenotype data. FIG. 27A: Multidimensional scaling (MDS) plot. Unstimulated, freshly isolated NK cells are used as controls. Arrows indicate the transitions across timepoints of experiment. FIG. 27B: Uniform Manifold Approximation and Projection (UMAP) plot was generated based on the arcsinh-transformed expression of the 17 expression markers in the NK cells from the whole dataset. Cells are colored according to the 31 clusters generated after manually merging the 40 meta-clusters obtained with FlowSOM. FIG. 29C: Individual UMAP plots of different NK cell conditions. Different time points indicated on the left side (baseline, 12 h, D10).

FIG. 28 (includes FIGS. 28A-28C): Expression of NK cell receptor ligands on target and NK cells. FIG. 28A: Gating strategy. FIG. 28B: Heatmap of the percent (%) expression of indicated ligands on MV-4-11 cells or (FIG. 28C) NK cells at each experimental time point.

FIG. 29 Comparisons of mean percent (%) cytotoxicity between different NK cell conditions in the serial stimulation assay. Each cell is subdivided to represent days 1-10 as indicated in upper left corner. Every cell's value signifies the p-value of each comparison for every day. P values generated with ordinary 2-way ANOVA corrected for multiple comparisons using the method of Bonferroni.

FIG. 30 (includes FIGS. 30A-30C): Transcriptomic evaluation of individual genes and differential expression analysis of IL15 secreting NKs. FIG. 30A: Volcano plot representing upregulated and downregulated genes in 2B4.ζ/sIL15 compared to sIL15 NK cells. Orange dots on the left represent downregulated and dots on the right represent upregulated genes. Location of each data point is calculated as log 2(FC)×−log 10(p-value). Color cutoffs: p-value <0.05; fold change cutoff>2 or <½. FIG. 30B: Bar plot depicting the top 10 significant pathways enriched in the differentially expressed genes. KEGG 2021 Human gene Set Enrichment Analysis was used. Dotted line represents the cutoff p-value of 0.05. FIG. 30C Dot plots displaying the normalized expression of individual genes (n=3 independent donors). Statistical significance: *p<0.05; **p<0.01; ***p<0.001; ****p<0.0001.

FIG. 31 (includes FIGS. 31A-31D): IL15 stimulated NK cells promote lethal toxicity of MV-4-11 engrafted mice. FIG. 31A: Schematic of MV-4-11 xenograft treated with IL15-secreting NK cells. On day 0, NSG mice were injected via tail vein with 1×106 CD123(+) MV-4-11 cells. In treatment groups (2B4.z/sIL15 or sIL15), 10×106 NK cells were administered on day 7. FIG. 31B: Kaplan-Meier survival analysis of MV-4-11 xenografts (n=5 mice each group). Dotted lines border grey shading representing usual survival window of MV-4-11 xenograft model. FIG. 31C: Mouse peripheral blood (PB) was collected at indicated time points and analyzed via flow cytometry. NK cell numbers per microliter of mouse PB were tracked starting on day 14 of the experiment. Each dot represents cell numbers from a single mouse. FIG. 31D: Human IL15 from peripheral blood of MV-4-11 engrafted mice at the indicated time points was quantified (pg/mL) with ELISA (mean+/−SEM, each dot representative of a single mouse).

FIG. 32 (includes 32A-32E): IL15 stimulation promotes NK cell expansion and inflammation in vivo. FIG. 32A: FACS plots of hCD45(+) cells in the peripheral blood, bone marrow, and spleen of mice at necropsy. hCD45(+)CD33(−)GFP(−) cells: NK cells (blue). Percentage of cells populating NK or AML gates indicated. FIG. 32B: Percentage (%) cell populations in spleen and bone marrow at necropsy (n=3 mice). Mean+/−SEM. FIG. 32C: Human TNFα. FIG. 32D: mouse IL1 and FIG. 32E mouse IL6 from peripheral blood of MV-4-11 engrafted mice drawn at necropsy and quantified with ELISA. Each dot derived from a single mouse. Mean+/−SEM.

FIG. 33 (includes FIGS. 33A-33D): IL15-secreting CAR-NK cell treatment promotes inflammation in MOLM-13 engrafted mice. FIG. 33A Human IL15, FIG. 33B human TNFα, FIG. 33C mouse IL1 and FIG. 33D mouse IL6 analysis from the peripheral blood drawn at necropsy from MOLM-13 engrafted mice. Cytokine measurement (pg/mL) with ELISA. Each dot represents data from a single mouse. n=2-5 mice.

FIG. 34 (includes FIGS. 34A-FIG. 34E). Truncation of CD3ζ chain distally after the first ITAM does not alter CAR expression or CAR-NK cell cytotoxic capacity. FIG. 34A: Schema of the CAR with CD3ζ truncated after the first ITAM (represented in small boxes). FIG. 34B: Percentage (%) of CAR+NK cells in cultures on D8 (black circle) and D18 (white circle). n=4 donors. FIG. 34C: Short-term percent (%) cytotoxicity of NK cells. Co-culture assays were performed using Raji (CD123−), MV-4-11 and MOLM-13 (CD123+) cancer cell lines. Raji cells engineered to express CD123 were also used. NK cells were cultured for 24 h at indicated effector:target (E:T) ratios with target cells expressing firefly Luciferase (ffLuc). Bioluminescence (BL) was measured following addition of D-luciferin and was compared to control condition without effector cells as an indicator of target cell death. n=4 donors; For the above assays, every bar (B) or every point (C) represent the mean value plus standard error of mean (SEM) for every NK condition. FIG. 34D: Heat map of the percent (%) NK cell cytotoxicity. Each column represents the specific day and each row the respective NK cell condition. Each square is color coded according to the % cytotoxicity and represents one replicate of each donor. Dark blue represents the lowest and bright yellow the highest value; n=6 (2 replicates of each of 3 donors). FIG. 34E: NK cell counts in the serial stimulation assay over a period of 10 days. Initial seeding count was 2 million NK cells.

FIG. 35 (includes FIGS. 35A-35D). IL-15 secreting CAR-NKs with truncated CD3ζ chain also exhibit dramatic in vivo expansion and lethal toxicity. 1 million MV-4-11 cell were injected via tail vein and a single dose of 3 million 2B4.ζ(Δ)/sIL15 was administered 4 days after. FIG. 35A: Leukemia proliferation was monitored with bioluminescence imaging and was measured as average radiance (photons/sec/cmÂ²/sr) (n=5 mice per condition). FIG. 35B: Mouse peripheral blood (PB) was collected at indicated time points and analyzed via flow cytometry. NK cell numbers per ul (microliter) of mouse PB were tracked over time starting on day 13 of the experiment. Each dot represents cell numbers from a single mouse. The line connects the median for every condition in different time points. FIG. 35C: Kaplan-Meier survival analysis of MV-4-11 xenografts (n=5 mice). FIG. 35D: Human IL15 from peripheral blood of MV-4-11 engrafted mice drawn at the indicated time points was quantified (pg/mL) with ELISA. Every dot represents values for a single mouse. Mean+/−SEM; n=1-3 mouse per day. Statistical significance: * p<0.05; ** p<0.01; ***p<0.001; ****p<0.001.

FIG. 36 (includes FIGS. 36A-36E). Mutation of the two distal ITAMS of the CD3ζ chain does not alter CAR expression or CAR-NK cell cytotoxic capacity. FIG. 36A: Schema of the CAR with the 2 mutated distal ITAMS of CD3ζ (X). Site directed mutagenesis was used to mutate the relevant tyrosines (Y60, Y72, Y91, and Y102) to phenylalanine to prevent phosphorylation. FIG. 36B: Percentage (%) of CAR+NK cells in cultures on D8 (black circle) and D18 (white circle). n=3 donors. FIG. 36C: Short-term percent (%) cytotoxicity of NK cells. Co-culture assays were performed using Raji (CD123−), MV-4-11 and MOLM-13 (CD123+) cancer cell lines. Raji cells engineered to express CD123 were also used. NK cells were cultured for 24 h at indicated effector:target (E:T) ratios with target cells expressing firefly Luciferase (ffLuc). Bioluminescence (BL) was measured following addition of D-luciferin and was compared to control condition without effector cells as an indicator of target cell death. n=3 donors; Bar (B) or Point (C) represent the mean value plus standard error of mean (SEM) for each NK condition. FIG. 36D: Heat map of the percent (%) NK cell cytotoxicity. Each column represents the specific day and each row the respective NK cell condition. Each square is color coded according to the percent cytotoxicity and represents one replicate of each donor. Dark blue represents the lowest and bright yellow the highest value; n=3 donors FIG. 36E: NK cell counts in serial stimulation assay over a period of 10 days. Initial seeding count was 250,000 NK cells (dotted line); mean+/−SEM, n=3 donors.

### DETAILED DESCRIPTION

Provided herein are, inter alia, methods, compositions and kits for treating and preventing cancer, e.g., acute myeloid leukemia. An engineered cell including a chimeric antigen receptor (CAR) polypeptide comprising a hinge domain, a transmembrane domain, and/or an intracellular domain is described. The engineered cell includes an immune cell, wherein the immune cell comprises natural killer (NK) cells or T cells. In embodiments, the immune cell comprises NK cells.

Advantages of the invention include: high and stable chimeric antigen receptor expression on NK cells, enhanced target (cancer, AML)—specific activation of CAR-NK cells, powerful target-specific cytotoxicity of CAR-NK cells against disease (AML), improved NK cell survival and persistence with engineered product, sustained specific anti-tumor activity of CAR-NK cells engineered to secrete cytokine. Other advantages include the use of NK cells verusus T cells. NK cells do not cause allogeneic toxicity, so they can be transferred from healthy donors. Unmodified NK cells when tested ex vivo have powerful anti-AML cytotoxicity but have not shown antitumor efficacy in clinical trials. This is likely because they do not persist and are not activated “enough” post-transfer. The capability to engineer in the CAR, with stable long-term expression, that confers more powerful, targeted anti-tumor activity, and then to stimulate increased longevity with the IL-15 secretion are likely to lead to aa clinical benefit, at least because of the data provided herein.

Combinations of various NK cell activating and stimulatory domains were evaluated with alternate hinge and transmembrane domains in the generation of CAR-NK cells. These CAR structures are unique, and they were evaluated for stability and density of surface expression, stimulation of specific anti-tumor activity, and in vivo performance. Moreover, the engineered cells were tested in a xenograft model of human acute myeloid leukemia. Peripheral blood in the mice was sampled, and inadequate circulating NK cell persistence (survival) to control/cure disease long-term was observed. Thus, constitutive secretion of the cytokine interleukin-15 was engineered in, which is known for NK cell activation and stimulated survival (and tested in clinical trial of CAR-NK cells targeted vs. CD19). Enhanced anti-tumor activity and survival was observed in the cell-based assays, and mouse models are tested.

Unique CAR constructs were generated with mutated immunoreceptor tyrosine-based activation motif (ITAM) domains, which has been shown to be optimal in CAR-T cells, but not published in CAR-NK. NK cells express both FcεR1γ and the CD3ζ activating domains to transduce intracellular signals through the NKG2D activating receptor. FcεR1γ encodes a single ITAM-like domain to propagate downstream signal, while CD3ζ encodes three ITAMs. Unexpectedly, the proximal CD3ζ ITAM alone provides a superlative activating signal (when compared to intact FcεR1γ or the full CD3ζ) to promote antigen-specific NK cell cytotoxicity without stimulating antigen-induced cell death (AICD) when expressed as a component of a single chain CAR. Cytokine secretion was evaluated in CAR-NK cells engineered to also constitutively express cytokines. This stimulated enhanced activity and persistence.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989).

Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation (e.g., acute myeloid leukemia) has occurred, but symptoms are not yet manifested.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (cutaneous T-cell lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with cancer, e.g., acute myeloid leukemia, and compared to samples from known conditions, e.g., a subject (or subjects) that does not have cancer, e.g., acute myeloid leukemia, (a negative or normal control), or a subject (or subjects) who does have cancer, e.g., acute myeloid leukemia, (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have cancer, e.g., acute myeloid leukemia, in a healthy or general population. The amount of a compound can be measured in a test sample and compared to the “normal control” level, utilizing techniques such as reference limits, discrimination limits, or risk defining thresholds to define cutoff points and abnormal values (e.g., for cancer, e.g., acute myeloid leukemia, or a symptom thereof). The normal control level means the level of one or more compounds or combined compounds typically found in a subject known not suffering from cancer, e.g., acute myeloid leukemia, Such normal control levels and cutoff points may vary based on whether a compounds is used alone or in a formula combining with other compounds into an index. Alternatively, the normal control level can be a database of compounds patterns from previously tested subjects who did not develop cancer, e.g., acute myeloid leukemia, or a particular symptom thereof (e.g., in the event the cancer, e.g., acute myeloid leukemia, develops or a subject already having cancer, e.g., acute myeloid leukemia, is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine. and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-f-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (s-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982))

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

### Acute Myeloid Leukemia (AML)

Acute myeloid leukemia (AML) is a cancer of the myeloid line of blood cells, characterized by the rapid growth of abnormal cells that build up in the bone marrow and blood and interfere with normal blood cell production. Symptoms may include feeling tired, shortness of breath, easy bruising and bleeding, and increased risk of infection. Occasionally, spread may occur to the brain, skin, or gums. As an acute leukemia, AML progresses rapidly and is typically fatal within weeks or months if left untreated.

First-line treatment of AML consists primarily of chemotherapy, and is divided into two phases: induction and postremission (or consolidation) therapy. The goal of induction therapy is to achieve a complete remission by reducing the number of leukemic cells to an undetectable level; the goal of consolidation therapy is to eliminate any residual undetectable disease and achieve a cure. Hematopoietic stem cell transplantation is usually considered if induction chemotherapy fails or after a person relapses, although transplantation is also sometimes used as front-line therapy for people with high-risk disease. Efforts to use tyrosine kinase inhibitors in AML continue.

AML has poor outcomes. The standard risk disease has about a 70% survival with very intensive chemoetherapy, in contrast, high risk or relapsed patients have about a 20% survival. Overall, about 40% of children with AML die of the disease, and in adults, about 27.4% survive about 5 years with the disease.

### CD19-CAR T Cells can Cure Chemoresistant Acute Lyphocytic Leukemia

Current problems with CAR-T development in AML include target selection, durability of anti-tumor response is dependent of infused T cells, and patient T cells post-chemotherapy regimens are not fit for expansion or engineering.

NK Cells Vs. AML

While NK cells are the first response in innate activity against viruses and malignancy, AML blasts express activating stress ligands. NK cells do not cause graft versus host disease (GVHD), and allogenic therapy is possible or preferred for AML. NK cells do not have a “usual” long-lived memory, whereas targeting of AML-associated antigen on normal myeloid cells is possible. In AML, the microenvironment is rich for NK survival, and the first lymphocyte subset to recover post-transplant are NK cells. Past allogenic NK adoptive transfer has been shown safe, but has not led to durable disease control.

### Methods of Producing the Engineered Cell Comprising the CAR Construct

Provided herein are methods of producing an engineered cell (e.g., an NK cell) comprising a CAR. For example, CAR transgenes were designed and generated by linking the CD123-specific scFV (26292) sequence to the hinge, transmembrane and intracellular moieties of the indicated activation and costimulatory molecules. Sequences were synthesized (GeneArt, ThermoFisher Scientific) and subcloned into pSFG retroviral vectors. CD3ζ mutants (ITAMIXX and ITAM1STOP) were generated using the QuikChange II XL Site-Directed Mutagenesis Kit (Agilent Technologies, Palo Alto, CA) and Gibson Assembly. Primers for mutagenesis were obtained from Integrated DNA Technologies. All sequences were validated by Sanger Sequencing (Johns Hopkins Genetic Resources Core Facility).

Healthy donor peripheral blood mononuclear cells (PBMCs; Anne Arundel Medical Blood Donor Center, Annapolis, MD; Carter Bloodcare, Woodway, TX) were isolated by ficoll centrifugation and CD3+ cells depleted using CD3-microbeads (Militenyi Biotec, Cologne, Germany). Remaining cells were stimulated on day 0 with lethally irradiated K562 feeder cells expressing membrane bound IL15 and 4-1BB ligand at a 1:1 ratio and cultured in SGCM media (CellGenix, Freiburg, Germany) with 10% Fetal Bovine Serum and 2 mmol/L Glutamax. NK cell purity was verified with flow cytometry using CD56-BV421 (318328; BioLegend) and CD3-PE (555340; BD) antibodies, NK cells were maintained and expanded in 200 IU/mL of recombinant human interleukin (IL)-2 (Biological Resources Branch Preclinical Biorepository, National Cancer Institute, Frederick, MD). NK cells were transduced on day 4 using transiently produced replication incompetent RD 114 pseudotyped retroviral particles immobilized on RetroNectin (Clontech Laborotories, Palo Alto, CA). The retroviral particles were generated in 293T cells by transfection of Peq-PAM, RD 114 and vector plasmids CAR expression was confirmed with flow cytometry on days 4 and 14 post-transduction.

### Methods for Treating Cancer, e.g., Acute Myeloid Leukemia (AML)

Included herein is a method of preventing or treating cancer, e.g., acute myeloid leukemia, in a subject in need thereof. In further embodiments, the method comprises administering to the subject an effective amount of the composition comprising an engineered cell (e.g., an NK cell) comprising a CAR. For example, methods for preventing or treating cancer, e.g., acute myeloid leukemia, include administering a composition comprising an engineered cell (e.g., an NK cell) comprising a CAR.

In other embodiments, the methods for treating cancer, e.g., acute myeloid leukemia, comprise administering to a subject a composition comprising an engineered cell (e.g., an NK cell) comprising a CAR produced according to the methods described herein, in combination with methods for controlling the outset of symptoms. In particular, the combination treatment can include administering readily known treatments. Additionally, combination therapy may include hormonal and/or chemotherapy (e.g. taxane-based) treatment (therapy). In embodiments, the combination therapy may include administration of a composition comprising an engineered cell (e.g., an NK cell) comprising a CAR

The described composition can be administered as a pharmaceutically or physiologically acceptable preparation or composition containing a physiologically acceptable carrier, excipient, or diluent, and administered to the tissues of the recipient organism of interest, including humans and non-human animals.

The composition (e.g., a composition comprising an engineered cell (e.g., an NK cell) comprising a CAR) can be prepared by re-suspending in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising an engineered cell (e.g., an NK cell) comprising a CAR) is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

In embodiments, a therapeutically effective amount of the composition (e.g., a composition comprising an engineered cell (e.g., an NK cell) comprising a CAR) in humans can be any therapeutically effective amount. In one embodiment, the composition (e.g., a composition comprising an engineered cell (e.g., an NK cell) comprising a CAR) is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., an engineered cell (e.g., an NK cell) comprising a CAR) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

Additional advantages of the methods described herein include that the composition (e.g., a composition comprising an engineered cell (e.g., an NK cell) comprising a CAR) can be injected systemically, as opposed to local delivery. Additional advantages include that patients requiring treatment typically require at least 1 local injections, and the injections are about 7 days apart. The compositions and methods described herein provide that patients require about 1 injection(s), systemically. In some examples, the injections can be every week.

### Pharmaceutical Compositions and Formulations

The present invention provides pharmaceutical compositions comprising an effective amount of a composition (e.g., a composition comprising an engineered cell (e.g., an NK cell) comprising a CAR) and at least one pharmaceutically acceptable excipient or carrier, wherein the effective amount is as described above in connection with the methods of the invention.

In one embodiment, the composition (e.g., a composition comprising an engineered cell (e.g., an NK cell) comprising a CAR) is further combined with at least one additional therapeutic agent in a single dosage form. In one embodiment, the at least one additional therapeutic agent comprises chemotherapy, radiation therapy, arsenic trioxide therapy, immunotherapy, or stem cell therapy. Other additional therapies include antibody therapy, antibody drug conjugate therapy, bispecific antibody therapy, immune checkpoint blocking agent therapy, or cytokine delivery.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits Comprising the Engineered Cell Comprising the CAR

In aspects, a kit for producing an engineered cell variant is provided. In embodiments, the kit comprises the engineered CAR and reagents.

In embodiments, the engineered cell comprising the CAR in the kit is suitable for delivery (e.g., local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a cancer, e.g., AML), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: CAR Expression on NK Cells

### Cell Lines

HEK293T (human embryonic kidney), Raji (Burkitt's lymphoma) and MV-4-11 (myelomonocytic leukemia) cell lines were purchased from the American Type Culture Collection (ATCC, Manassas, VA) and cultured in Dulbecco's Modified Eagle's Medium (DMEM; ThermoFisher Scientific Waltham, MA; 293T), Roswell Park Memorial Institute (RPMI; ThermoFisher Scientific; Raji) or Iscove's Modified Dulbecco's Medium (IMDM; ThermoFisher Scientific; MV-4-11) supplemented with 10% fetal bovine serum (FBS; HyClone, Logan, UT). MOLM-13 cell line was purchased from the Leibniz Institute (DSMZ, German Collection of Microoganisms and Cell Cultures) and cultured in RPMI supplemented with 2 mmol/L L-glutamine (ThermoFisher Scientific). CD123 expressing Raji cells (Raji.CD123) were created by first subcloning the full length human CD123 coding sequence into a pCDH lentiviral backbone. Vesicular stomatitis virus G glycoprotein (VSV-G) pseudotyped lentiviral particles were produced according to the manufacturer's instructions. Subsequently Raji cells were transduced with the lentivirus. CD123-positive cells were isolated using fluorescence-activated cell sorting (FACS) and expression verified prior to use. All cells used for BLI based cytotoxicity analysis as well as in the xenograft model were transduced with a retroviral vector carrying an enhanced green fluorescent protein (GFP) firefly luciferase fusion gene 1(GFP.ffLuc). GFP-positive cells were sorted and maintained in the appropriate culture medium. Luciferase expression was confirmed using D-luciferin and quantification of bioluminescence. All cells were cultured in a humidified atmosphere containing 5% CO₂ at 37° C.

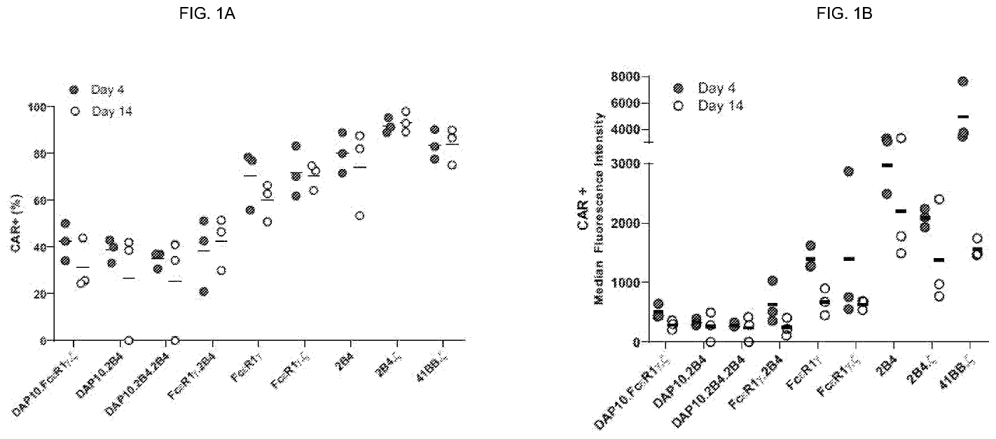

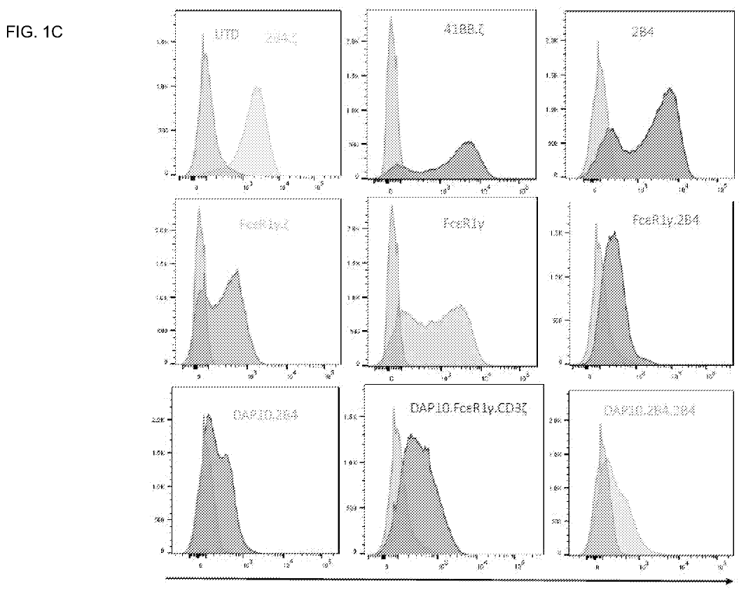

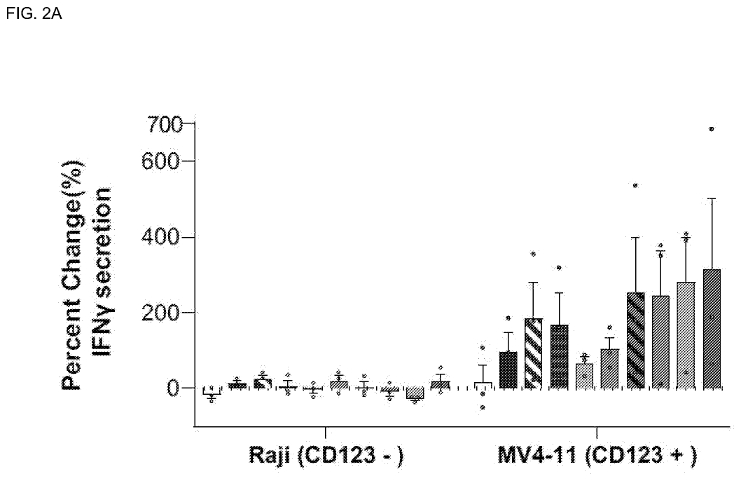

CAR transgenes were designed and generated by linking the CD123-specific scFV (26292) sequence to the hinge, transmembrane and intracellular moieties of the indicated activation and costimulatory molecules. Sequences were synthesized (GeneArt, ThermoFisher Scientific) and subcloned into pSFG retroviral vectors. CD3ζ mutants (ITAMIXX and ITAMISTOP) were generated using the QuikChange II XL Site-Directed Mutagenesis Kit (Agilent Technologies, Palo Alto, CA). Primers for mutagenesis were obtained from Integrated DNA Technologies. All sequences were validated by Sanger Sequencing (Johns Hopkins Genetic Resources Core Facility). Healthy donor peripheral blood mononuclear cells (PBMCs; Anne Arundel Medical Blood Donor Center 3, Annapolis, MD; Carter Bloodcare, Woodway, TX) were isolated by ficoll centrifugation and CD3+ cells depleted using CD3-microbeads (Militenyi Biotec, Cologne, Germany). Remaining cells were stimulated on day 0 with lethally irradiated K562 feeder cells expressing membrane bound IL15 and 4-1BB ligand 4 at a 1:1 ratio and cultured in SGCM media (CellzGenix, Freiburg, Germany) with 10% Fetal Bovine Serum and 2 mmol/L GlutamsD3-PE (555340; BD) antibodies, NK cells were maintained and expanded in 200 IU/mL of recombinant human interleukin (IL)-2 (Biological Resources Branch Preclinical Biorepository, National Cancer Institute, Frederick, MD). NK cells were transduced on day 4 using transiently produced replication incompetent RD 114 pseudotyped retroviral particles immobilized on RetroNectin (Clontech Laborotories, Palo Alto, CA). The retroviral particles were generated in 293T cells by transfection of Peq-PAM, RD 114 and vector plasmids 5. CAR expression was confirmed with flow cytometry on days 4 and 14 post-transduction.) CAR expression analysis was performed using incubation with His-tagged recombinant CD123 protein (SinoBiological, Beijing, China) and secondary staining with His-PE or His-APC (BioLegend, San Diego, CA). See, e.g., FIGS. 1A-1C.

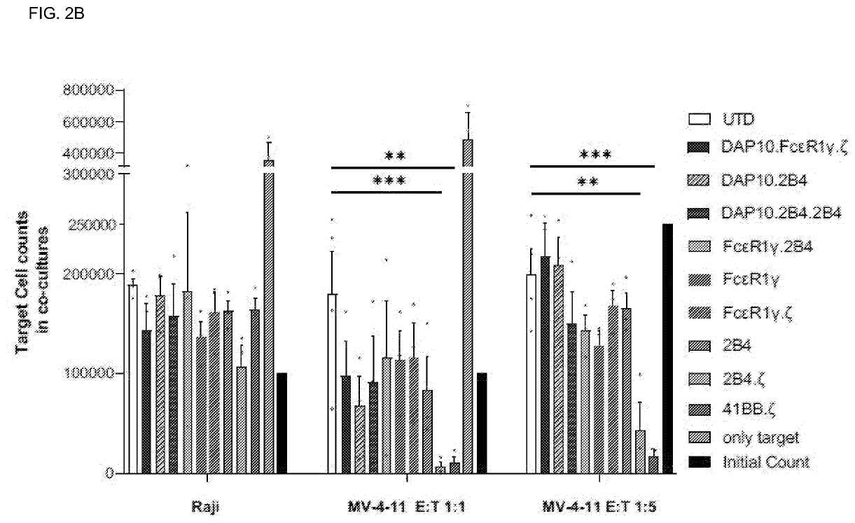

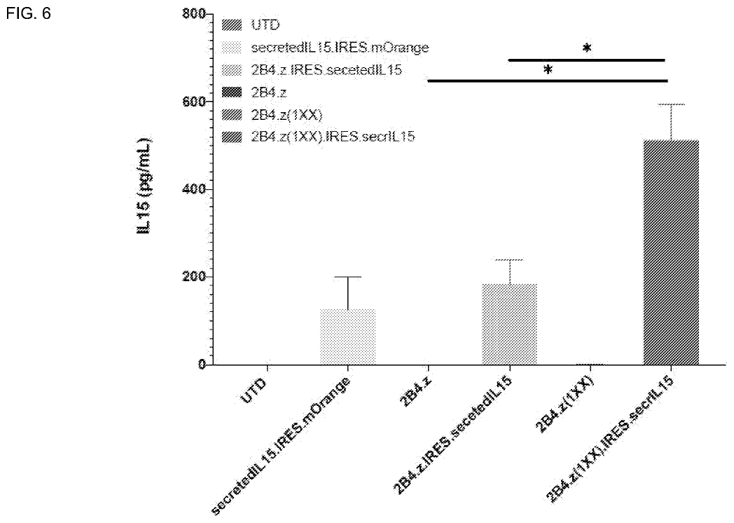

CAR-NK Cell Anti-Tumor Activation and Cytotoxicity, Measurement of IL15 Secretion/Enhanced Short-Term Cytotoxicity with Engineered IL15 Secretion (FIGS. 2A-2B, FIG. 4, FIG. 6, and FIGS. 7A-7C).

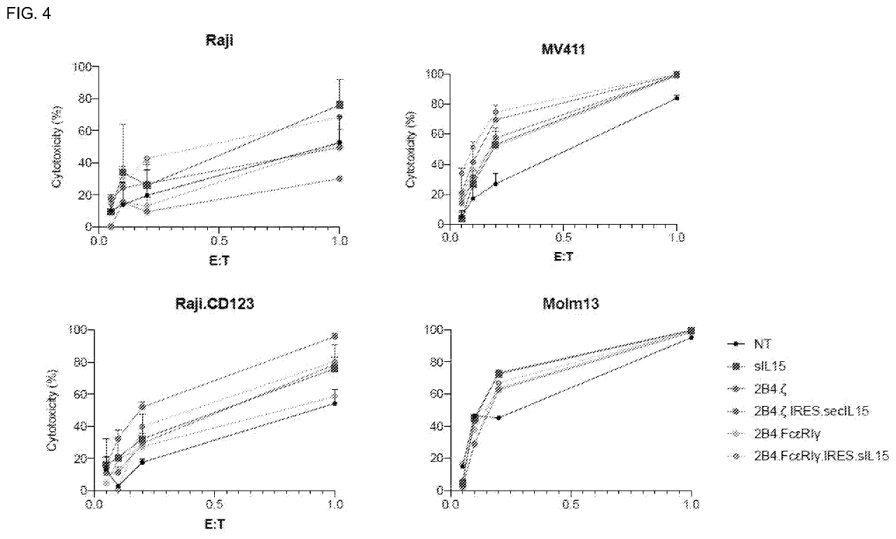

100,000 NK cells were plated with target cells in 0.2 mL media at a 1:1 E:T ratio for 24 hours. Supernatant was collected and IFNγ quantification performed via ELISA (R&D Systems, Minneapolis, MT). For measurement of IL15 secretion, 1 million NK cells were plated in 2 mL media. After 24 hours, supernatant was collected and IL15 quantification was performed with ELISA (R&D Systems) according to the manufacturer's instruction. % change in co-cultures (X) compared to no target (NK cell only) cultures (Y) was calculated with 100*(X-Y)/(Y). Different assays were used to evaluate NK cell cytotoxicity. A short-term co-culture of NK cells with target cells expressing enhanced green fluorescent protein (GFP) firefly luciferase fusion gene (GFP.ffLuc) in 1:1, 1:5, 1:10 and 1:20 E:T ratios (FIG. 4). 24 h later the bioluminescence was measured after addition of D-luciferin. Mean percentage of specific lysis of triplicate samples was calculated as 100*(spontaneous death-experimental death)/(spontaneous death-background). Spontaneous death was measured with control wells containing only target cells. NK cells were incubated with target cells for 3 days (FIGS. 2A and 2B). The cell count of the remaining NK cells and target cells was measured through flow cytometric analysis including CD56-BV421 (BD Biosciences, Franklin Lakes, NJ) CD33-PerCP-Cy5.5 (MV-4-11) and CD19-PerCP-Cy5.5 (BD Biosciences; Raji). Dead cells were excluded from analysis using LIVE/DEAD Fixable Viability Stain 780 (BD Horizon). Flow cytometric analysis of cell numbers was performed using CountBright™ counting beads (ThermoFisher).

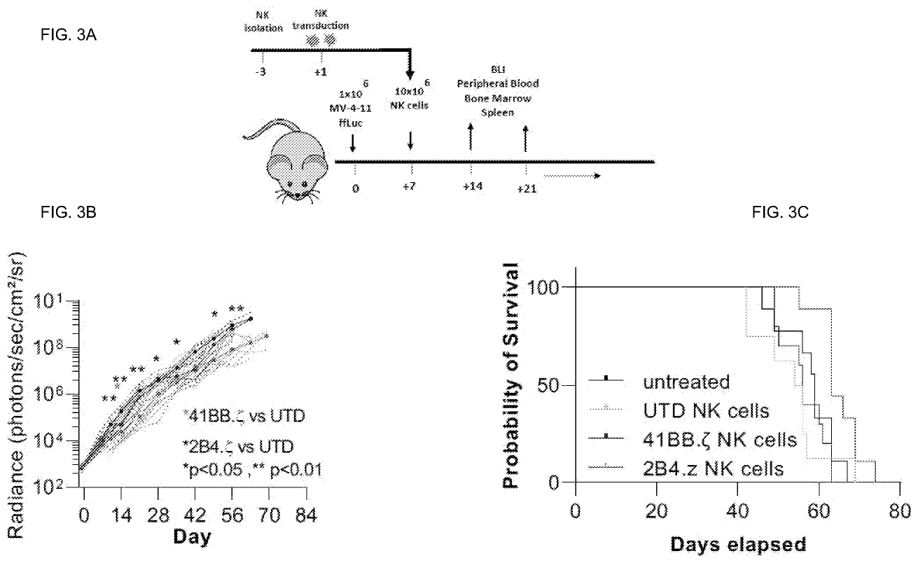

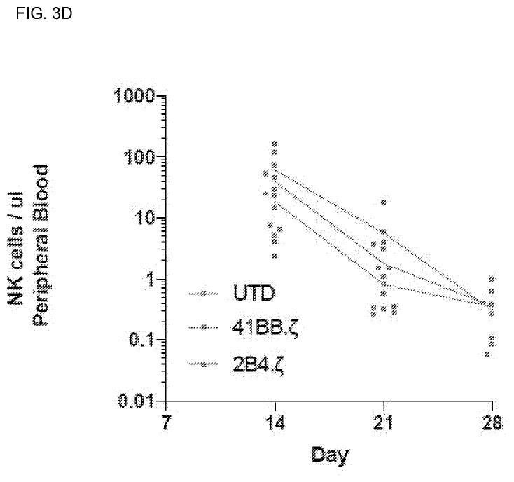

CAR-NK Cell In Vivo Anti-Tumor Activity and Persistence (FIGS. 3A-3D).

All animal studies were carried out under protocols approved by the Johns Hopkins Institutional Animal Care and Use Committee (IACUC). Six to eight week old female NSG (NOD scid gamma) mice were engrafted with 1e6 MV-4-11.ffLuc via tail vein injection. Ten million unmodified or CAR-transduced NK cells were injected one week after leukemia administration. Mice were given D-Lucifern (3 mg) by intraperitoneal injection and bioluminescence measured with the IVIS system. Data was analyzed using Living Image. Peripheral blood was drawn via facial vein and cells analyzed with flow cytometry. Bone marrow and spleen were harvested and tissues analyzed using flow cytometry. Mice were euthanized when they exhibited >20% weight loss, hind limb paralysis or moribund state.

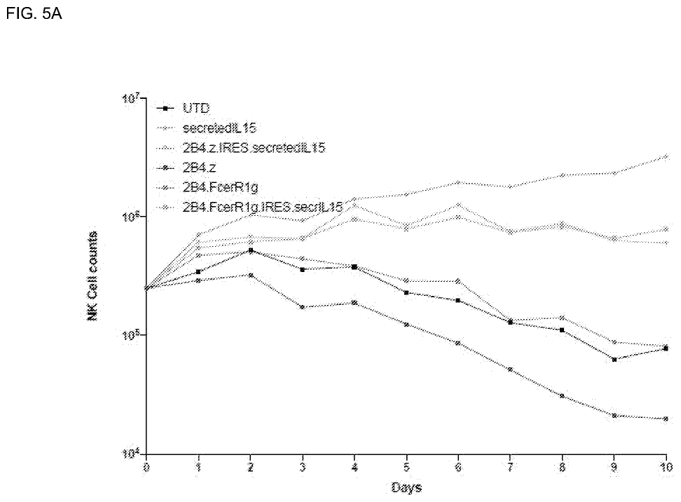

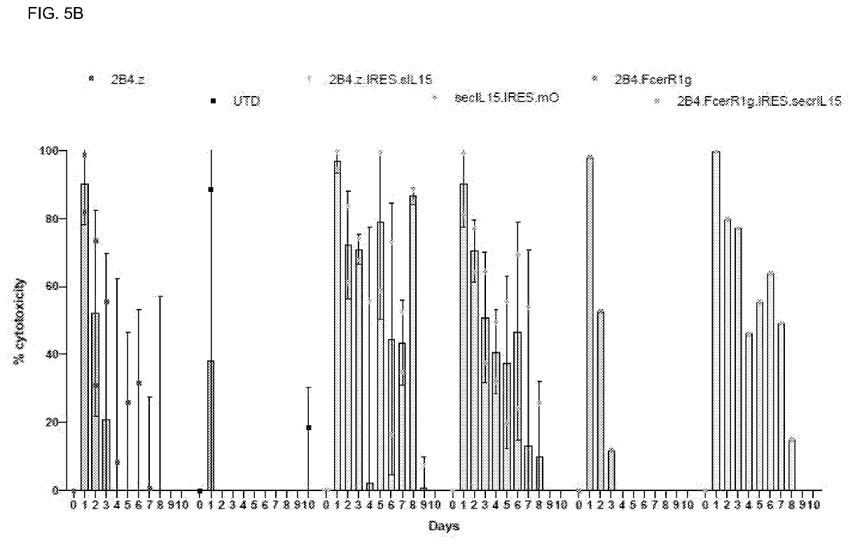

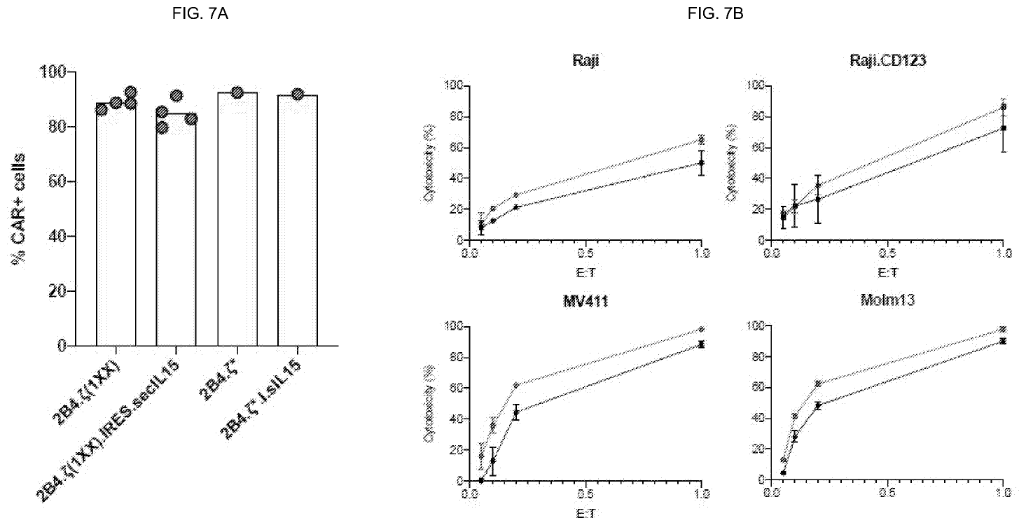

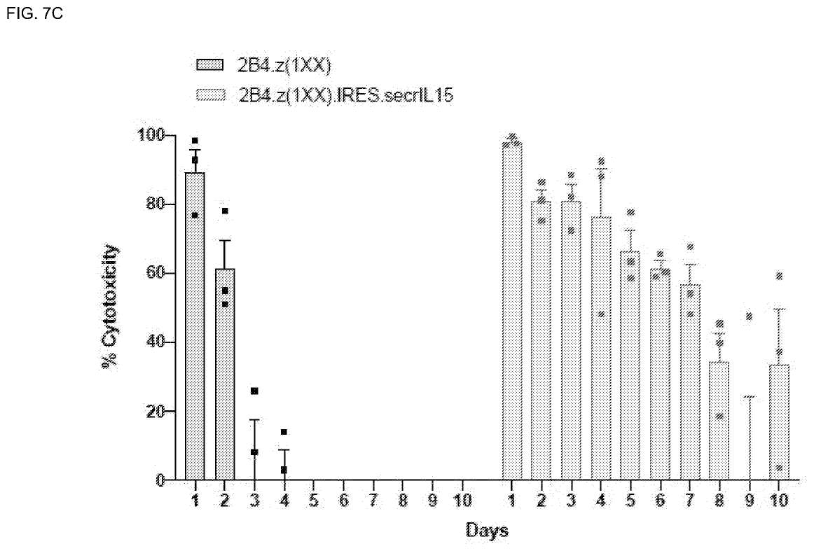

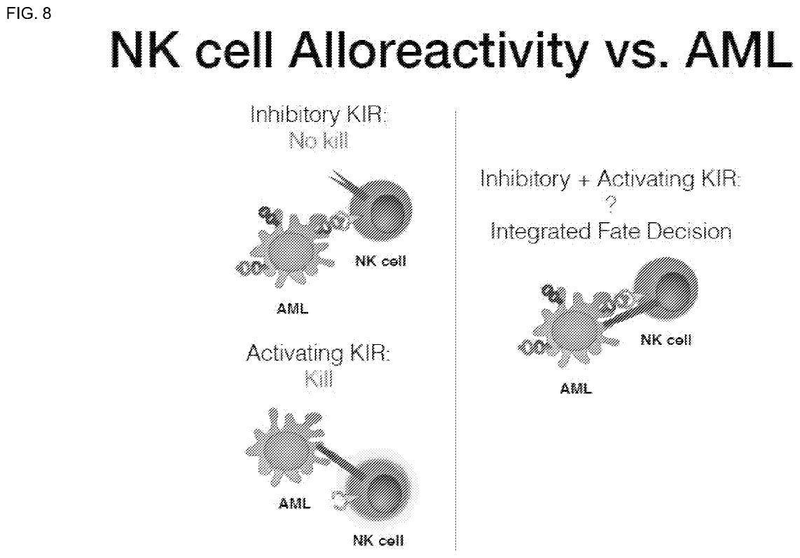

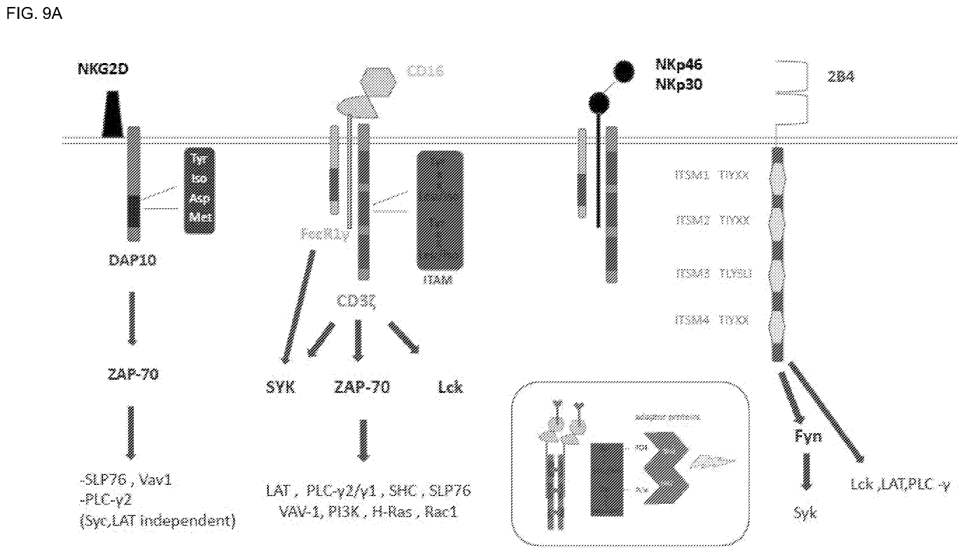

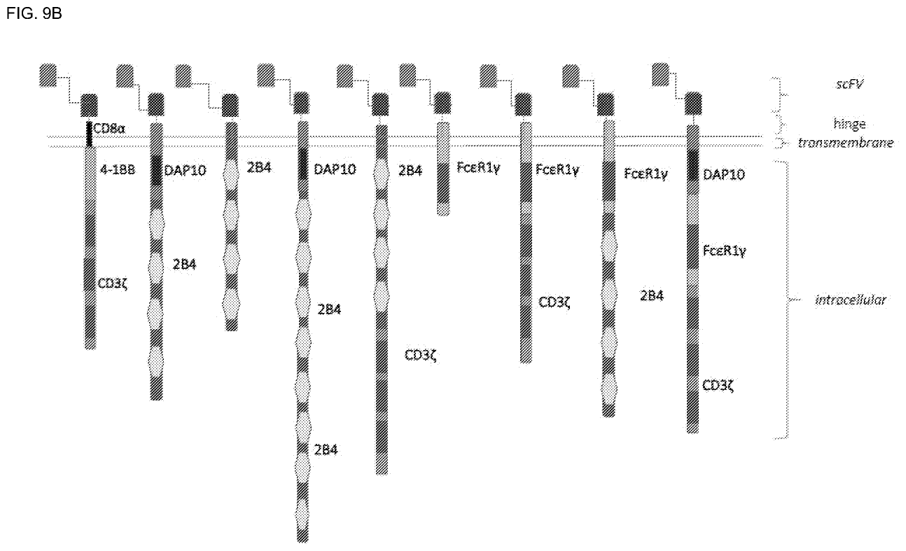

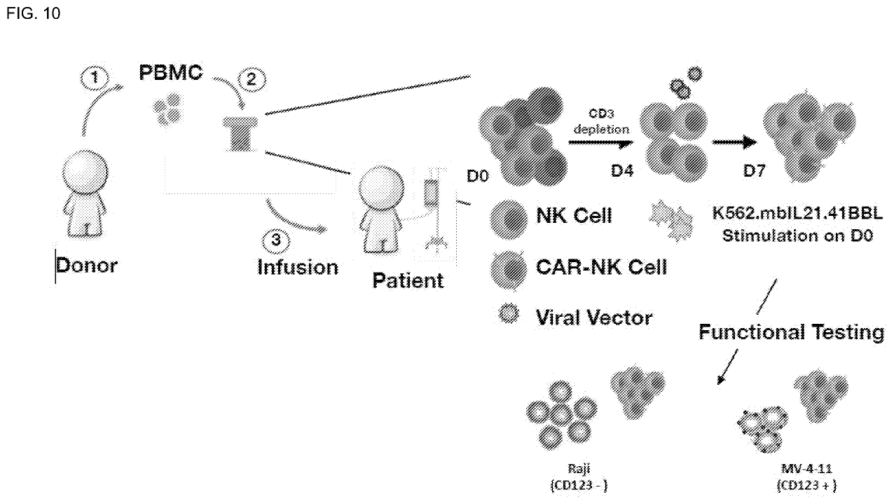

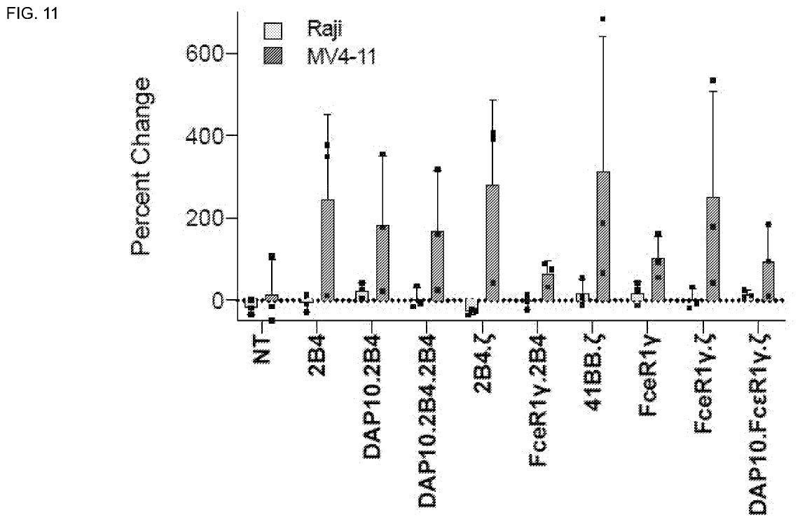

Engineered Interleukin-15 (IL15) secretion improves CAR-NK persistence and cytotoxicity in model of chronic antigen exposure (FIGS. 5A-5B and FIG. 7C).

NK cells were stimulated in 1:1 E:T ratio daily for a total of ten days with MV-4-11 cells. NK cell proliferation and cytotoxicity was measured through flow cytometric analysis of remaining NK and target cells in the culture. NK cells stimulated with 200 IU/ml of IL-2 every two days were used as controls. % cytotoxicity was calculated based on the target cell numbers on the day of stimulation (Y) and the day after (X) based on the formula 100*(X-Y)/(Y).

Statistical Analysis

All analyses was carried out using GraphPad Prism Software (v8). For comparisons of 2 groups, 2-tailed unpaired t tests were used. Two-way analysis of variance (ANOVA) corrected for comparison using the method of Sidek was used for comparison of 3 or more groups. Survival of mice was estimated by the Kaplan-Meier method and differences in survival between groups were calculated by the log-rank (Mantel-Cox) test. p<0.05 was considered as statistically significant.

### Example 2

### Methods

### Chimeric Antigen Receptor (CAR) Generation

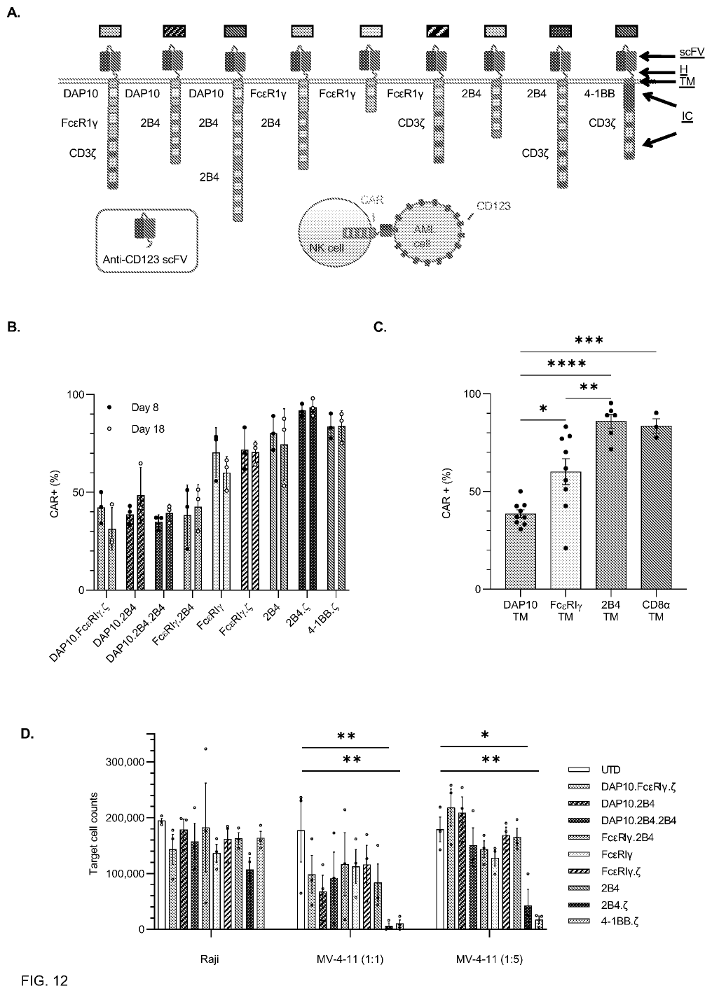

CAR transgenes were designed using the CD123-specific single chain variable fragment (scFv; 26292)²⁵ sequence and the hinge, transmembrane, and intracellular domains indicated in FIG. 12A. Sequences were synthesized (GeneArt, ThermoFisher Scientific) and subcloned into pSFG retroviral vectors. All sequences were validated by Sanger Sequencing (Johns Hopkins Genetic Resources Core Facility).

### CAR-NK Cell Production

Healthy donor peripheral blood mononuclear cells (PBMCs; Anne Arundel Medical Blood Donor Center, Annapolis, MD; Carter Bloodcare, Woodway, TX) were isolated by Ficoll desnity gradient centrifugation and depleted of T cells using CD3-microbeads (Militenyi Biotec, Cologne, Germany). Remaining cells were stimulated on day 0 with lethally irradiated K562 feeder cells²⁶ expressing membrane bound IL15 and 4-1BB ligand at a 1:1 ratio. Cells were maintained in SCGM media (CellGenix, Freiburg, Germany) supplemented with 10% Fetal Bovine Serum, 2 mmol/L GlutaMAX (ThermoFisher), and 200 IU/mL hIL-2 (Biological Resources Branch Preclinical Biorepository, National Cancer Institute, Frederick, MD). NK cell purity was verified with flow cytometry using fluorophore conjugated antibodies against CD56 and CD3 (Table 1 below). NK cells were transduced on day 4 of culture using transiently produced replication incompetent RD 114 pseudotyped retroviral particles immobilized on RetroNectin (Clontech Laborotories, Palo Alto, CA).

Flow cytometry Antibodies for NK and cancer cell identification targeted CD56 and CD33 (AML cell lines) or CD19 (Raji) markers. A detailed list of all antibodies, including those used for the evaluation of immune cell phenotype is in Table 1 below. CAR expression analysis was performed using incubation with His-tagged recombinant CD123 protein (SinoBiological, Beijing, China) and secondary staining with αHis-PE or αHis-APC (BioLegend, San Diego, CA). Dead cells were excluded from analysis using LIVE/DEAD Fixable Viability Stains 780 or 575V (BD Horizon, Franklin Lakes, NJ). Cell enumeration was performed with CountBright™ counting beads (ThermoFisher). Human Fc receptors (FcRs) were blocked using Human TruStain FcX™ (BioLegend). In samples stained with multiple BD Horizon Brilliant reagents, Brilliant Stain Buffer Plus (BD Horizon) was used. Compensation was performed with UltraComp eBeads Compensation Beads (ThermoFisher). Cell surface antigens were quantified using microspheres of the Quantum APC Molecules of Equivalent Soluble Fluorochrome (MESF) kit (Bangs Laboratories, Inc.; Fisher, IN). All samples were acquired on FACSCelesta or FACSymphony Cell Analyzers (BD) and analyzed with FlowJo software (v10.6.1; v10.7.2). Cell sorting was performed on FACSMelody (BD).

TABLE 1
Fluorophore-conjugated antibodies used in flow cytometry
experiments.

| | | | Source |
|---|---|---|---|
| Marker | Fluorophore | Clone | (Company) |
| CD56 | BV421 | HCD56 | BioLegend |
| CD56 | APC | HCD56 | BioLegend |
| CD56 | BUV805 | NCAM16.2 | BD |
| CD16 | BUV563 | 3G8 | BD |
| CD33 | PerCP/Cyanine5.5 | P67.6 | BioLegend |
| CD33 | BV785 | WM53 | BioLegend |
| His-tag | APC | J095G46 | BioLegend |
| His-tag | PE | J095G46 | BioLegend |
| CD158b | BUV661 | CH-L | BD |
| (KIR2DL2/2DL3/ | | | |
| 2DS2) | | | |
| CD158e1 | BV480 | DX9 | BD |
| (KIR3DL1) | | | |
| CD158 | APC/Fire ™ 750 | HP-MA4 | BioLegend |
| (KIR2DL1/S1/ | | | |
| S3/S5) | | | |
| NKG2A (CD159a) | UV737 | 131411 | BD |
| NKp30 (CD337, | BB700 | p30-15 | BD |
| NCR3) | | | |
| NKp46 (CD335, | BB515 | 9E2/NKp46 | BD |
| NCR1) | | | |
| CD57 | BV605 | QA17A04 | BioLegend |
| CD62L | Red 718 | SK11 | BD |
| PVRIG | Alexa Fluor 405 | 2334A | R&D |
| | | | Systems |
| TIGIT | BUV395 | 741182 | BD |
| CD96 | BV711 | 6F9 | BD |
| NKG2D (CD314) | PE-Cy7 | 1D11 | BioLegend |
| DNAM-1 (CD226) | PE/DazzleTM 594 | 11A8 | BioLegend |
| TRAIL (CD253) | BV711 | RIK-2 | BD |
| FAS-L ( CD178) | BV421 | NOK-1 | BioLegend |
| TIM-3 (HAVCR2) | BB700 | 7D3 | BD |
| LAG3 (CD223 ) | BV650 | 11C3C65 | BioLegend |
| PD-1 (CD279) | BB515 | EH12.1 | BD |
| NKG2C (CD159c) | BV605 | 134591 | BD |
| 2B4 (CD244) | PE-CF594 | 2-69 | BD |
| KLRG1 (MAFA) | PE-Cy7 | 2F1/KLRG1 | BioLegend |
| CD69 | BUV805 | FN50 | BD |
| LFA-1 (CD11a) | Red 718 | G-25.2 | BD |
| CD161 | BUV395 | HP-3G10 | BD |
| TRAIL-R1 | BV421 | S35-934 | BD |
| (CD261) | | | |
| FAS (CD95) | BB515 | DX2 | BD |
| MICA/MICB | PE-Cy7 | 6D4 | BioLegend |
| (PERB11) | | | |
| ULBP-1 | APC | 170818 | R&D |
| | | | Systems |
| CD48 | PE-CF594 | TÜ145 | BD |
| CD112 (Nectin-2) | PerCP/Cyanine5.5 | TX31 | BioLegend |
| CD155 (PVR) | V711 | TX24 | BD |
| galectin-9 | PE | 9M1-3 | BD |
| PD-L1 (CD274) | UV395 | MIH1 | BD |
| CD19 | PerCP/Cyanine5.5 | SJ25C1 | BioLegend |

### Immunophenotype Analysis

Two different panels (A and B) were used for evaluation of NK cell receptors and one panel (C) for receptor ligands. Data analysis of the multiparameter panels A and B was performed in R (v3.6.2). The median marker intensities were transformed using arcsinh (inverse hyperbolic sine) with cofactor 150.²⁷ Nonlinear dimensionality reduction on randomly selected 500 data points per sample of each panel was performed using uniform manifold approximation and projection (UMAP).²⁸ NK cell clusters were identified with the FlowSOM (v1.18.0) algorithm and 40 different metaclusters were generated per panel.²⁹ Subsequently, we manually merged hierarchically neighboring clusters similar in biology and median marker intensities. Panel A clusters do not correlate with the ones in panel B.

### Serial Stimulation Assay

NK cells were stimulated daily with MV-4-11 cells at a 1:1 effector:target (E:T) ratio in G-rex plates (Wilson Wolf, New Brighton, MN) for a total of ten days. NK cell proliferation and cytotoxicity was measured using flow cytometric analysis. Percent (%) cytotoxicity was calculated based on the target cell numbers on the day of (Y) and the day after (X) stimulation using the formula 100*(X-Y)/(Y). Cell phenotype was evaluated at baseline, on the first (12 h) and the tenth (D10) day.

### RNA Sequencing

On D10 of serial stimulation, co-culture was depleted first of dead cells using the Dead Cell Removal Kit and next of leukemia cells with CD33-microbeads (Militenyi). NK cell purity was verified with flow cytometry using fluorophore-conjugated CD56 and CD33 antibodies. RNA was extracted from NK cells using the RNeasy Mini Kit (Qiagen, Hilden, Germany) and RNAseq was performed. Differential expression analysis and statistical testing was performed using DESeq2 software.³⁰

### Xenograft Mouse Model

All animal studies were carried out under protocols approved by the Johns Hopkins Institutional Animal Care and Use Committee (IACUC). Six to eight week old NSG (NOD.Cg-Prkdc^{scid}Il2rg^{tm1Wjl}/SzJ mice were obtained from an internal colony that originated from the Jackson Laboratory (Bar Harbor, ME). Mice were injected with 1×10⁶ MV-4-11 cells modified for stable firefly Luciferase (ffLuc) expression³¹ or 5×10⁴ MOLM-13.ffLuc cells³² via tail vein on day 0. NK cell treatment was administered on D7 (10×10⁶ cells) or D4, 7, and 10 (3×10⁶ cells each). Mice were given D-Luciferin (3 mg) by intraperitoneal injection and bioluminescence (BL) measured using IVIS Spectrum (In Vivo Imaging System). Data were analyzed using Living Image Software (v 4.7.3; PerkinElmer, Waltham, MA). When indicated, peripheral blood was drawn via facial vein, red blood cells were lysed with eBioscience RBC Lysis Buffer (ThermoFisher) and the remaining cells analyzed with flow cytometry. Bone marrow and spleen were harvested and tissues analyzed with flow cytometry. Analysis of peripheral blood for cytokines (human (h) IL15, hTNFα, mouse (m) IL6, mIL1β) was performed with ELISA (R&D Systems). Mice were euthanized when they exhibited >20% weight loss, hind limb paralysis or moribund state as per protocol guidelines.

### Cell Lines

HEK293T (human embryonic kidney), Raji (Burkitt's lymphoma) and MV-4-11 (myelomonocytic leukemia) cell lines were purchased from the American Type Culture Collection (ATCC, Manassas, VA) and cultured in Dulbecco's Modified Eagle's Medium (DMEM; ThermoFisher Scientific Waltham, MA; 293T), Roswell Park Memorial Institute (RPMI; ThermoFisher Scientific; Raji) or Iscove's Modified Dulbecco's Medium (IMDM; ThermoFisher Scientific; MV-4-11) supplemented with 10% fetal bovine serum (FBS; HyClone, Logan, UT). MOLM-13 cell line was purchased from the Leibniz Institute (DSMZ, German Collection of Microoganisms and Cell Cultures) and cultured in RPMI supplemented with 10% FBS. CD123 expressing Raji cells (Raji.CD123) were created by first subcloning the full length human CD123 coding sequence into a pCDH lentiviral backbone. Vesicular stomatitis virus G glycoprotein (VSV-G) pseudotyped lentiviral particles were produced using the pPACKH1 HIV Lentivector Packaging Kit (System Biosciences, Palo Alto, CA) according to the manufacturer's instructions and used for Raji cell modification. CD123-positive cells were isolated using fluorescence-activated cell sorting (FACS) and antigen surface expression verified prior to use. All cells used for BLI-based cytotoxicity assays and/or our xenograft models were transduced with a retroviral vector carrying an enhanced green fluorescent protein (GFP) firefly luciferase fusion gene (GFP.ffLuc) (Vera, J., et al. T lymphocytes redirected against the kappa light chain of human immunoglobulin efficiently kill mature B lymphocyte-derived malignant cells. Blood 108, 3890-3897 (2006)) GFPpositive cells were sorted and maintained in the appropriate culture medium. Luciferase expression was confirmed using D-luciferin and quantification of bioluminescence. All cells were cultured in a humidified atmosphere containing 5% CO₂ at 37° C.

### Determination of Vector Copy Number (VCN)

Primer/probe-FAM was designed to the MMLV-derived psi present in pSFG and purchased from ThermoFisher Scientific. RNAseP primer/probe-VIC/TAMRA mix (Applied Biosystems #4403326) was used as comparison. Genomic DNA was isolated from CAR-NK cells and 25 ng used for amplification with TaqMan Universal PCR Mastermix (ThermoFisher) and the above primer/probe mixes on a C1000 Touch Thermal Cycler (Bio-Rad, Hercules, CA). The following amplification conditions were used: 50° C. for 2 minutes, 95 C for 10 minutes, 40 cycles of 95 C for 15 seconds, 60° C. for 1 minute. No-template, unmodified NK cells and a condition containing only plasmid were used as controls. Vector copy number calculation was performed using the 2^{-ΔCt} method. Kunz, A., et al. Optimized Assessment of qPCR-Based Vector Copy Numbers as a Safety Parameter for GMP-Grade CAR T Cells and Monitoring of Frequency in Patients. Mol Ther Methods Clin Dev 17, 448-454 (2020).

### Cytotoxicity Assay

Bioluminescence (BL) based: NK cells were co-cultured with target cells expressing ffLuc at the indicated E:T ratios. D-luciferin was added to plate and BL measured per well. Mean percentage of specific lysis of triplicate samples was calculated as 100*(spontaneous death-experimental death)/(spontaneous death-background). Spontaneous death was measured with control wells containing only target cells. Flow cytometric: NK cells were cultured with target cells. NK and target cell numbers were measured using flow cytometric analysis and NK or target-cell specific markers as above, with dead cell exclusion.

### Cytokine Secretion Assay

100,000 NK cells were plated with an equivalent number of target cells in 0.2 mL media and cultured for 24 hours. Supernatant was collected and IFN quantification performed via ELISA (R&D Systems, Minneapolis, MT). For measurement of IL15 secretion, 1 million NK cells were plated in 2 mL media. After 24 hours, supernatant was collected and cytokine quantification was performed with ELISA (R&D Systems) according to the manufacturer's instruction.

### Library Preparation and RNA Sequencing

For RNA sequencing experiments, RNA samples were converted to double stranded cDNA using the Ovation RNA-Seq System v2.0 kit (Tecan, Männedorf, Switzerland), which utilizes a proprietary strand displacement technology for linear amplification of mRNA without rRNA/tRNA depletion as per the manufacturer's recommendations. This approach does not retain strand specific information. Quality and quantity of the resulting cDNA was monitored using the Bioanalyzer High Sensitivity kit (Agilent) which yielded a characteristic smear of cDNA molecules ranging in size from 500 to 2000 nucleotides in length. After shearing 500 nanograms of cDNA to an average size of 250 nucleotides with the Covaris S4 (Covaris Inc., Woburn, MA) library construction was completed with the Truseq Nano kit (Illumina; San Diego, CA) according to the manufacturer's instructions. mRNA libraries were sequenced on an Illumina Novaseq 6000 instrument using 150 bp paired-end dual indexed reads and 1% of PhiX control. Reads were aligned to GRCh38 using rsem version 1.3.0 with the following options—star-calc-ci-star-outputgenome-bam-forward-prob 0.5.

### Statistical Analysis

All statistical analyses was performed using GraphPad Prism Software (v9.2.0). Our comparisons included more than three groups and ordinary one- or two-way analysis of variance (ANOVA) corrected using the method of Bonferroni. Data with variance of several logs of magnitude were log transformed (Y=log(Y)) before analyzing with ANOVA. Survival of mice was estimated by the Kaplan-Meier method and differences in survival between groups were calculated by the log-rank (Mantel-Cox) test.

### Results

### CD123-CARs are Highly Expressed on the NK Cell Surface

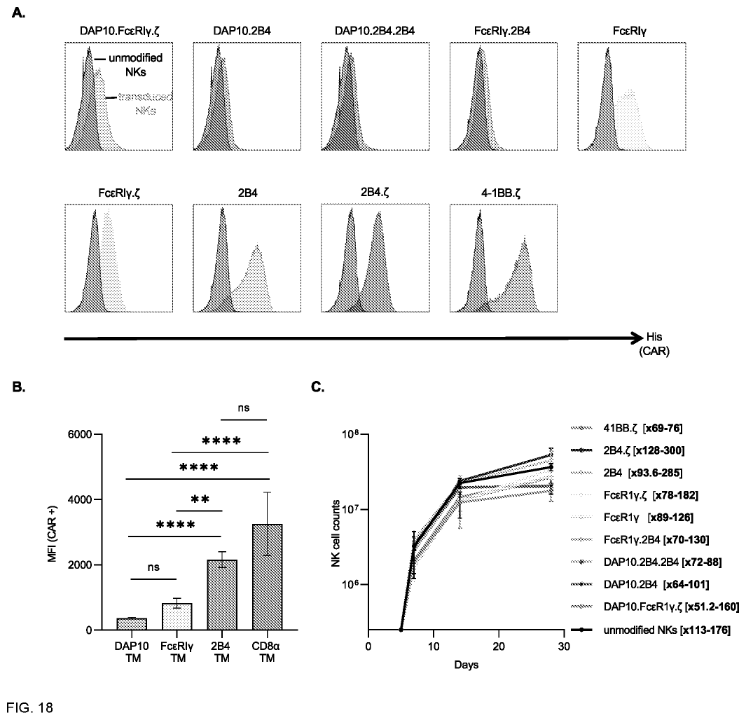

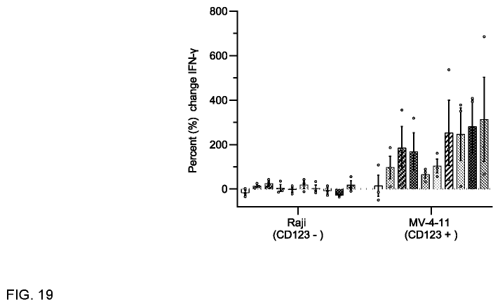

We considered NK cell biology in our design of 8 different NK-tailored CARs (FIG. 12A) to complement the common 4-1BB.ζ CAR.^{33,34} All CARs are comprised of an extracellular scFv targeting CD123.²⁵ The hinge, transmembrane (TM), and intracellular portion of our CARs consisted of different combinations of activating co-receptors DAP10 and FcεR1γ, the co-stimulatory receptor 2B4, and the ζ chain of the T cell receptor (FIG. 12A). All CARs were expressed stably on the surface of primary human NK cells for at least two weeks in culture, with transduction efficiencies ranging from 21-98% (FIG. 12B). Representative flow cytometric plots are shown in FIG. 18A. CARs encoding 2B4 or CD8α TM domains demonstrated higher transduction efficiencies (median(range), 89(53-98%) and 84(75-90%), respectively) than constructs containing FcεR1γ or DAP10 TM (62(21-77%) and 39(24-64%), respectively; FIGS. 12A and 12B). 2B4 and CD8α TM domains also conferred optimal CAR surface density as estimated by comparative mean fluorescence intensities (MFI+/−SEM; 2B4-TM: 2158+/−242; CD8α-TM: 3254+/−970; FcεR1γ-TM: 827+/−151; DAP10-TM: 366+/−23; 2B4 and CD8α vs DAP10: p<0.0001; 2B4 vs FcεR1γ: p<0.01; CD8α vs FcεR1γ: p<0.0001; FcεR1γ vs DAP10: ns; FIG. 18B). CAR-NK cells expanded 70-213 fold within 18 days of ex-vivo culture with no significant differences between generated CAR-NK cell populations (FIG. 18C).

CD123-CAR NK Cells have Antigen-Specific Anti-AML Activity In Vitro

We evaluated the target specificity of our CAR-NK cells using the CD123-positive MV-4-11 and CD123-negative Raji cell lines. When challenged with MV-4-11 in co-culture assays, all CAR-NK cells responded with enhanced cytokine secretion above that seen when using unmodified NK cells under identical conditions (mean percent (%) change of IFN-γ secretion; CAR-NKs(range): 65-313% vs unmodified: 14%, FIG. 19). There was no difference in cytokine production of CAR(+) and CAR(−) NK cells after co-culture with CD123(−) targets (FIG. 19). Next, we assessed CAR-NK cell cytotoxicity against CD123(+) target cells in 72-hour co-culture assays. We found that CARs with 2B4 or 4-1BB co-stimulatory and TCRζ signaling domains endowed NK cells with the greatest cytolytic activity against MV-4-11 at both 1:1 (mean % cytotoxicity+/−SEM; 2B4.ζ: 93.8+/−5%; 4-1BB.ζ: 89.9+/−6.8%) and 1:5 effector:target (E:T) ratios (82.9+/−11.6%; 93+/−2.6%, respectively; FIG. 12D). CD123(−) Raji cells were again used as controls. There was no difference between CAR-NK versus unmodified NK cell mediated cytotoxicity against Raji cells, confirming specificity (FIG. 12D).

CD123-2B4.ζ CAR-NK Cells have Limited Anti-AML Efficacy In Vivo

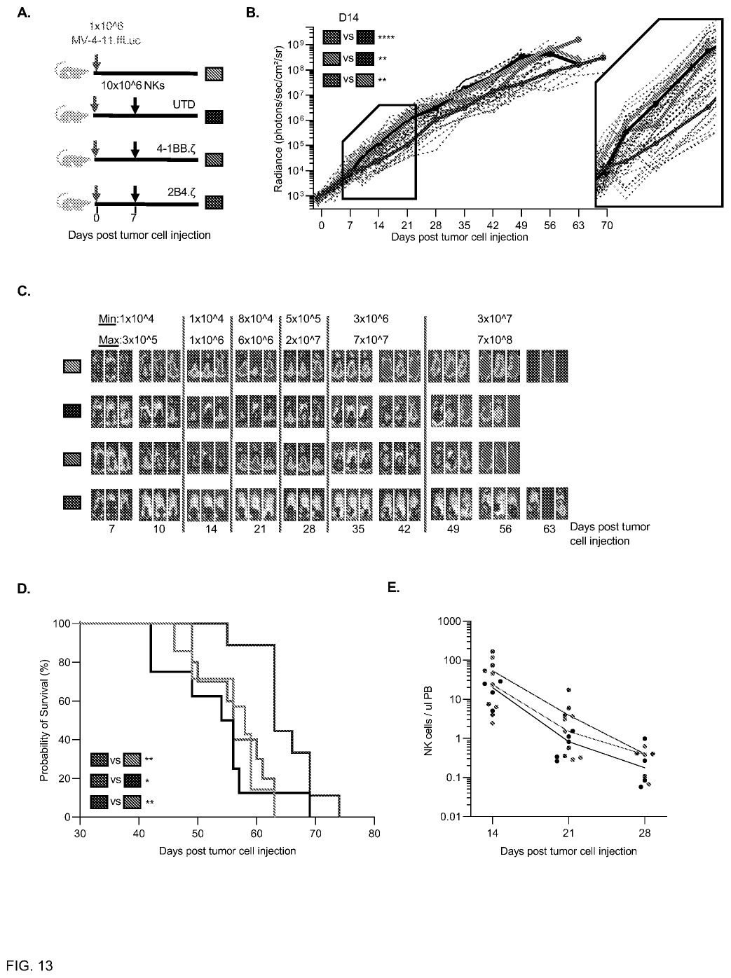

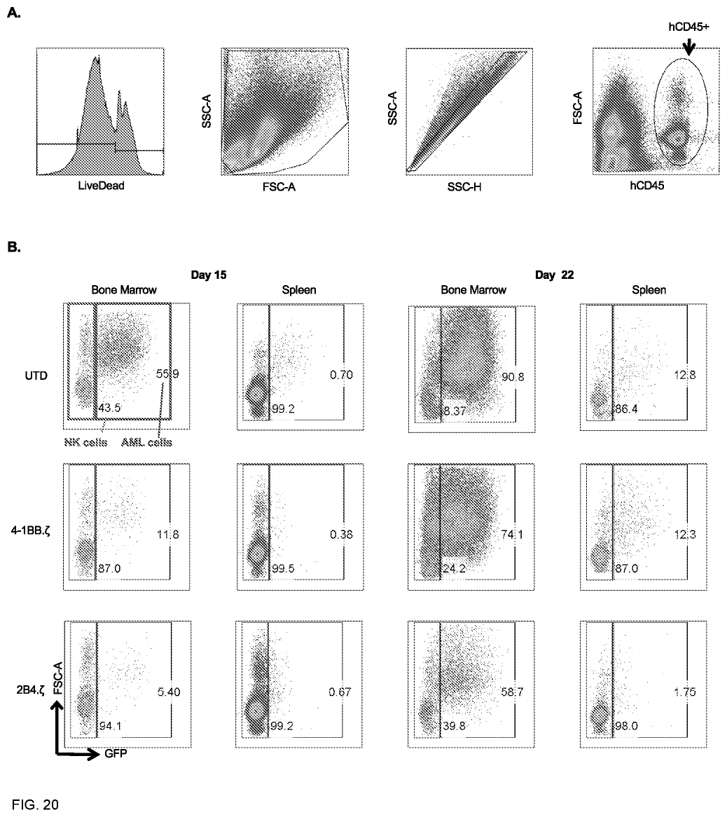

Given the superior anti-AML activity of 2B4.ζ and 4-1BB.ζ CAR-NK cells in our in vitro assays, we next evaluated their antitumor activity in a xenograft model of human AML. NSG mice were first engrafted with MV-4-11.ffLuc cells,³¹ then treated with CAR-NK or unmodified NK cells on day 7 (FIG. 13A). Leukemic growth was measured with serial BL imaging. 2B4.ζ CAR-NK cells post injection had transient anti-AML activity, which translated into a significant survival advantage in comparison to other experimental groups (median survival in days; 2B4.ζ vs 4-1BB.ζ; 63 vs 56, p<0.01; 2B4.ζ vs unmodified; 63 vs 55, p<0.05) and untreated controls (2B4.ζ vs no treatment; 63 vs 58, p<0.01; FIGS. 13B-D). Serial analysis of peripheral blood in our animals showed declining NK cell numbers in all evaluated mice irrespective of infused NK cell type (FIG. 13E). Planned bone marrow and spleen examination in a subset of mice demonstrated increasing percentages of leukemia from day 15 to day 22 in all treatment conditions, despite readily detectable NK cells (FIG. 20). Consistent with BL data, the percentage of leukemic cells was lower in both bone marrow and spleen samples of 2B4.ζ CAR-NK cell treated mice as compared to mice treated with UTD and 4-1BB.ζ CAR-NK cells (FIG. 20).

Armoring NK Cells with Secreted IL15 Enhances Anti-AML Functionality In Vitro

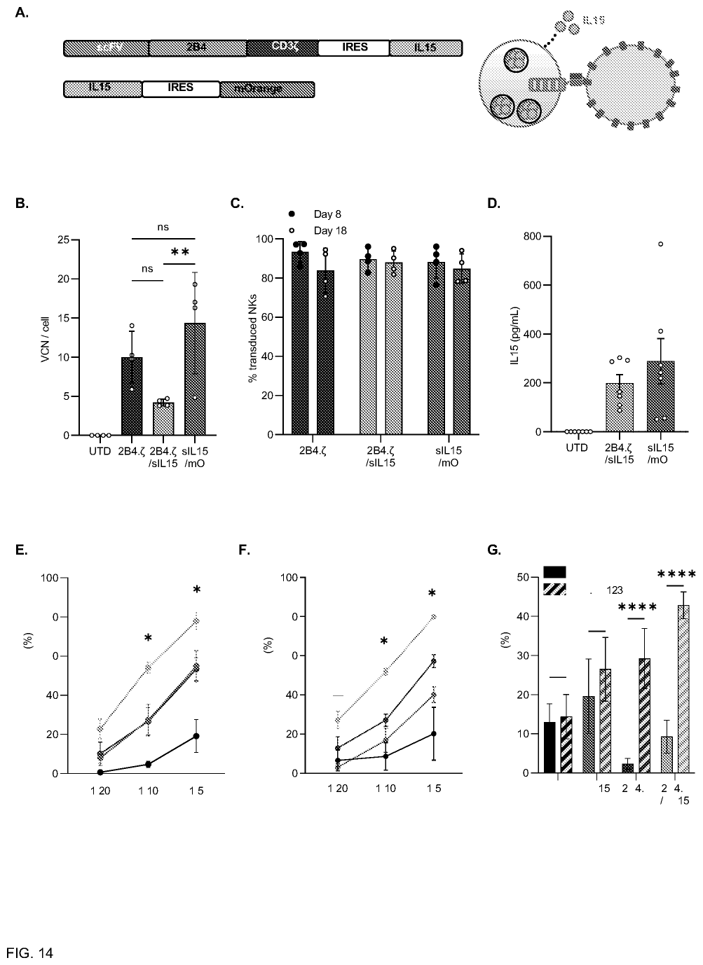

Having demonstrated that 2B4.ζ CAR-NK cells have anti-AML cytolytic capacity with short persistence, we next explored if transgenic expression of IL15 in CAR-NK cells would support sustained anti-AML activity. To accomplish this, we cloned a sequence encoding human IL15 downstream of an IRES element into our 2B4.ζ CAR vector. We simultaneously generated a second retroviral vector encoding IL15 and the fluorescent molecule mOrange as a control with IL15, but not CAR expression (FIG. 14A). We verified transduction by measuring vector copy number (VCN) per cell (median [range]; 2B4.ζ: 10[5.8-14]; 2B4.ζ/secretory(s)IL15: 4.1[3.8-4.7]; sIL15/mOrange: 16.6[4.8-19.2]; FIG. 14B).

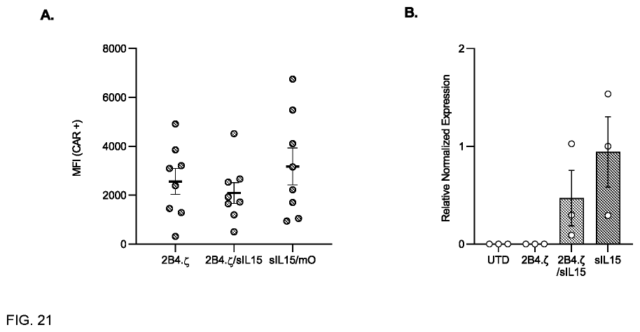

We found no significant difference in VCN/cell or in CAR expression between NK cells transduced with 2B4.ζ or 2B4.ζ/sIL15 encoding retroviral vectors (range CAR(+); 2B4.ζ vs 2B4.ζ/sIL15; 70-97% vs 81-96%, p>0.99; MFI+/−SEM; 2B4.ζ vs 2B4.ζ/sIL15; 2,566+/−531 vs 2,089+/−424, p>0.99; FIG. 14C; FIG. 21A). Expression of IL15 was measured with qRT-PCR (FIG. 21B) and IL15 secretion was confirmed by ELISA (FIG. 14D).

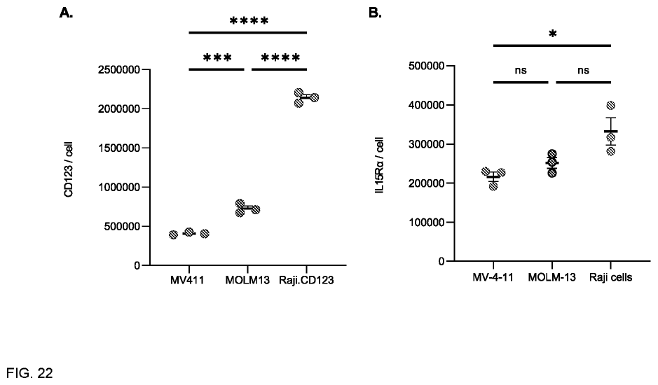

We evaluated short-term cytotoxicity of 2B4ζ/sIL15 CAR-NKs against the CD123(+) MV-4-11, MOLM-13, and Raji.CD123 cell lines. The parental CD123(−) Raji line was used as a negative control. When compared to 2B4.ζ, the 2B4ζ/sIL15 CAR-NKs had higher cytotoxicity against CD123+targets (FIGS. 14E-14G). Both 2B4.ζ and 2B4.ζ/sIL15 demonstrated antigen-specific cytotoxicity as Raji.CD123 were more effectively killed than parental (CD123 negative) Raji cells (FIG. 14G). Target cell CD123 and IL15Ra expression were quantified in order to evaluate for any effect of CD123 surface density or IL15 trans presentation on NK cell cytotoxicity (FIG. 22A). Differences in measured CD123 surface density did not correlate with observed short-term cytotoxicity, underlining the existence of additional complex mechanisms affecting NK cell activation. Similarly, differences observed in IL15Rα expression did not correlate with cytotoxicity (FIG. 22B).

### Transgenic Expression of IL15 Potentiates the Activation, Persistence, and Long-Term Cytolytic Activity of CAR-NK Cells

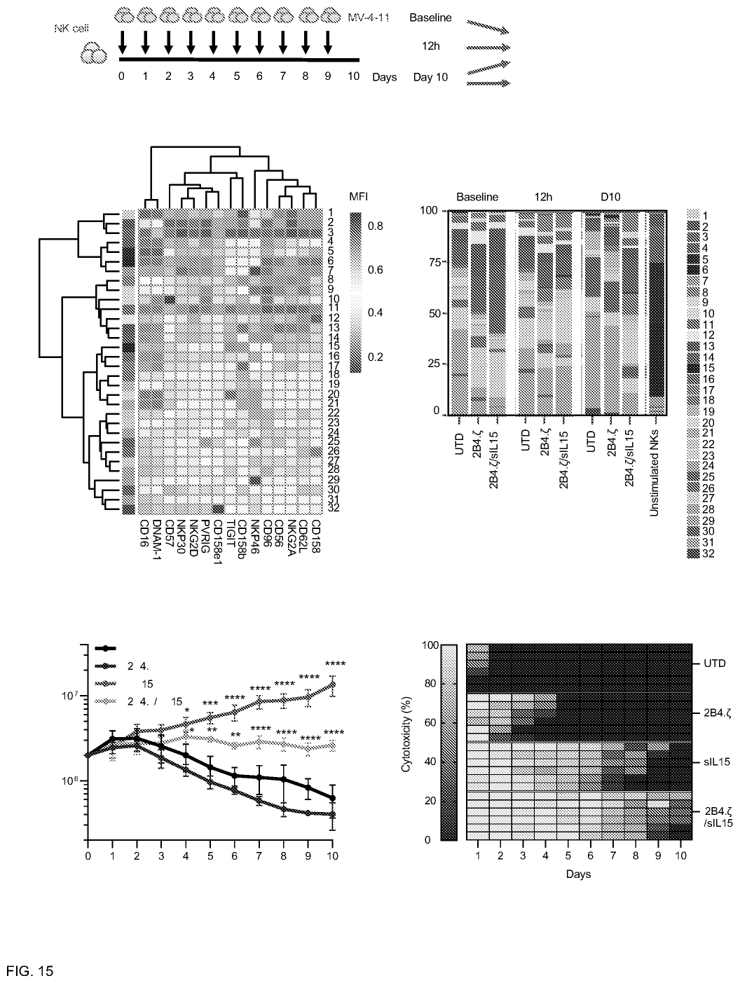

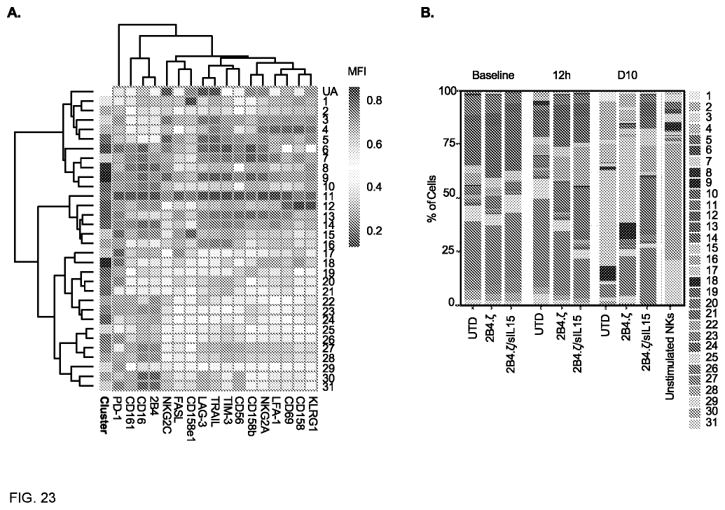

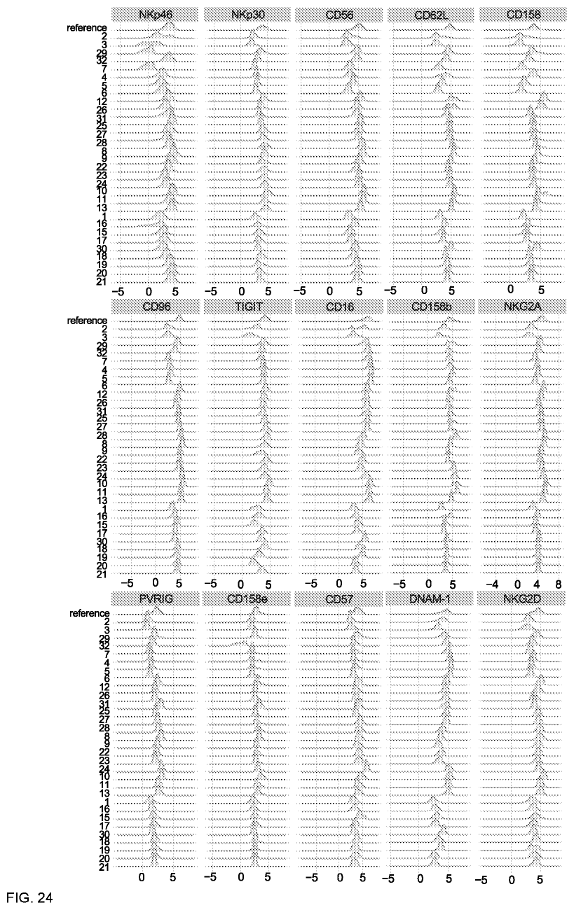

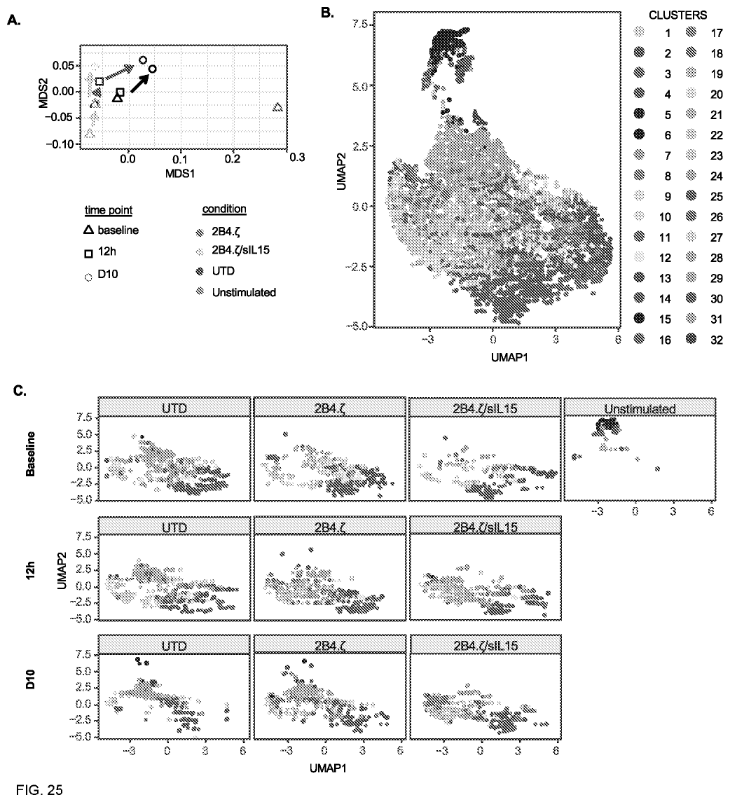

Using a model of chronic antigen stimulation (FIG. 15A), we evaluated the immune phenotype of our CAR-NK cells at baseline, after 12 hours, and on day 10 (D10) of co-culture with MV-4-11. Cells were counted daily, and AML repleted to maintain a 1:1 E:T ratio. We used flow cytometry to measure surface expression of markers in two different panels (panels A and B), then performed hierarchical clustering of NK cell subsets (FIG. 15B, 15C, FIG. 23). In panel A, we observed similar population distributions in the 2B4.ζ CAR and unmodified NK cells with cluster shifts from earlier (12 h) to later (D10) time points. Specifically on D10, there was an increase in the percentage of 2B4.ζ and unmodified NK cells populating clusters defined by lower surface expression of activating receptors NKG2D and NKp30 (clusters 19, 25, 31; FIG. 15B, 15C, FIG. 24). For 2B4.ζ/sIL15 CAR-NK cells, only minimal changes in population density of these subsets were observed on D10 (FIG. 15B, 15C). The unique 2B4.ζ/sIL15 immunophenotype is highlighted by comparison of the MDS (global) and UMAP (cluster-specific) plots in FIG. 25.

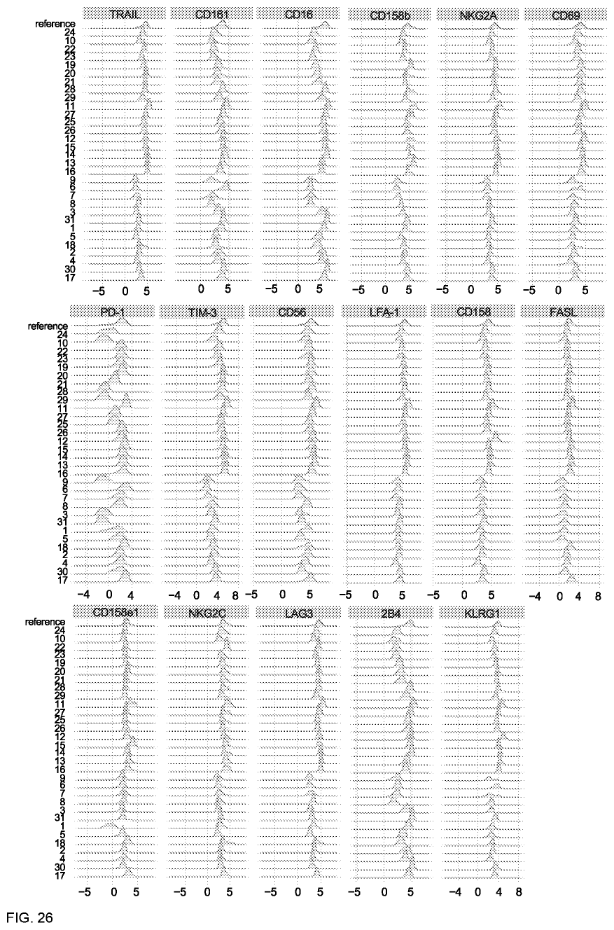

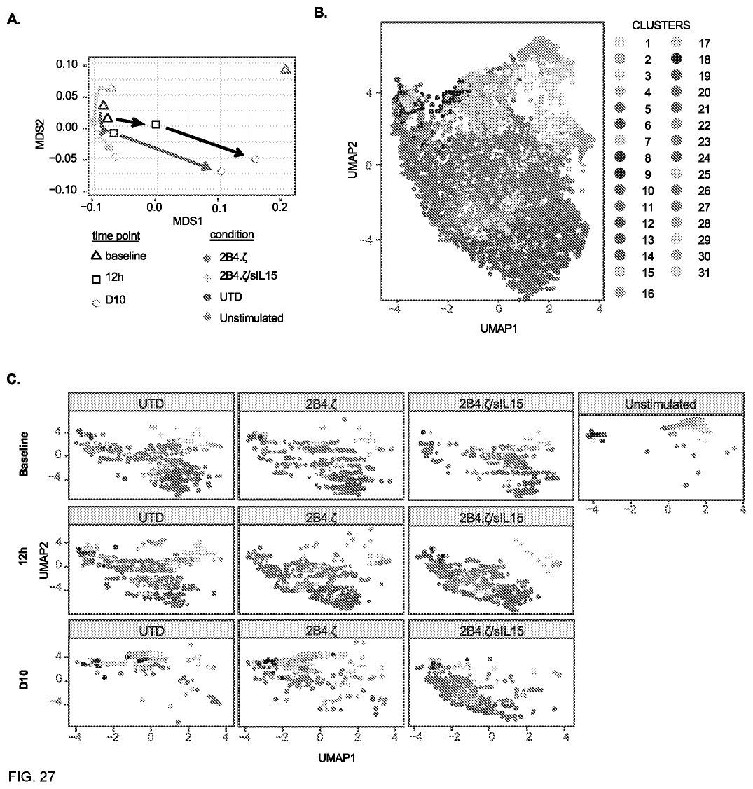

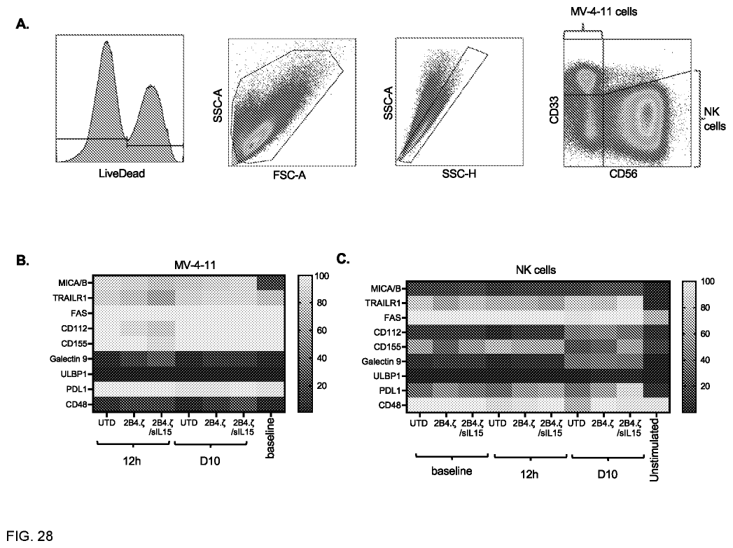

Clustering of NK cells based on expression of markers included in our second receptor panel (panel B) again supported the maintained NK cell activation to D10 in 2B4.ζ/sIL15 CAR-NK cells (predominant clusters 16, 20, 26 and minor clusters 11,13, 14, 15, 21, 23; FIG. 23, FIG. 26). Differences were again observed in 2B4.ζ and unmodified NK cells on D10 compared to earlier time points (increasing percentage of cells populating clusters 2, 4, 17, 18, and decreasing percentages of 14, 16, 20, 26; FIG. 23). Clusters 14, 16, 20, and 26 expressed higher levels of LFA-1, CD69, TRAIL, TIM-3, NKG2A, and KLRG1 compared to clusters 2, 4, 17, and 18 (FIG. 26). Negligible differences were observed in PD-1, LAG-3, FASL, 2B4, and NKG2C expression. Taken together, 2B4.ζ/sIL15 CAR-NKs had a higher percentage of NK cells populating clusters defined by higher surface expression of LFA-1(adhesion/activation receptor), CD69 (activation marker), TRAIL (death receptor), and TIM-3 (commonly upregulated after NK cell activation), as well as NKG2A and KLRG1 (inhibitory receptors; FIG. 15B, FIG. 15C, FIGS. 23-27). Overall, the data from both antibody panels suggests that with continuous antigen stimulation, IL15 preserves an activated CAR-NK cell phenotype associated with high cytotoxicity. We also analyzed the expression of NK cell activating and inhibitory ligands. AML cells expressed high levels of MICA/MICB (NKG2D ligands), CD112 and CD155 (DNAM-1, PVRIG, CD96, and TIGIT ligands) and PDL1 (PD-1 ligand), but not ULBP1 (NKG2D ligand) or galectin-9 (TIM-3 ligand; FIG. 28).

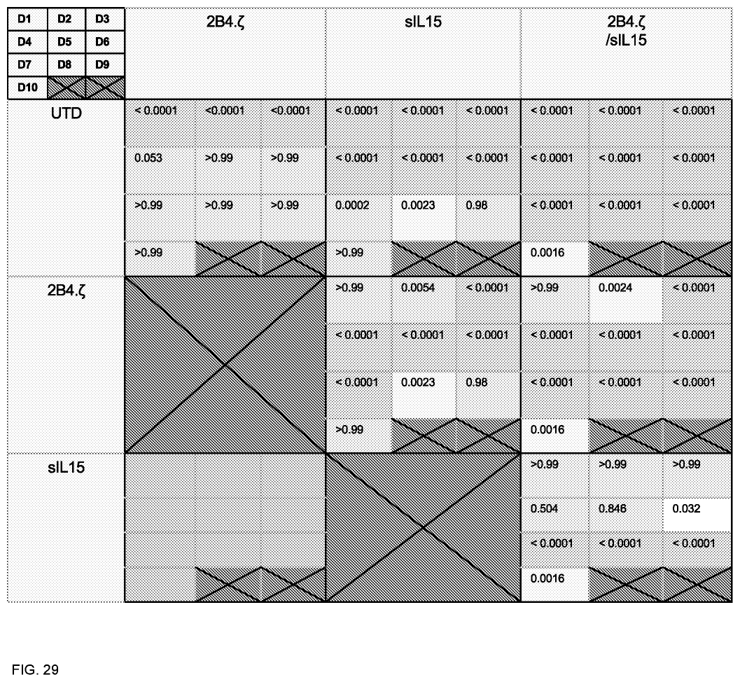

Next, we evaluated NK cell cytotoxicity and persistence in our model of chronic antigenic stimulation (FIG. 15A). While unmodified and 2B4.ζ CAR-NK cells sharply diminished in number over the course of this assay, NK cells genetically engineered to express IL15 (2B4.ζ/sIL15 and sIL15) survived throughout the experiment (n=3; FIG. 15D). As predicted by the short-term cytotoxicity assay, 2B4.ζ CAR-NK cells had high initial anti-AML activity. However, their killing potential decreased as 2B4.ζ CAR-NK cell counts declined. Cytotoxicity was completely abrogated by day 5 (FIG. 15E). IL15 activation alone (without CAR expression) also had an early anti-tumor effect that was not sustained. Only the combination of CAR expression and IL15 secretion led to continued NK cell mediated anti-AML cytotoxicity (FIG. 15E; FIG. 29).

NK Cells Secreting IL15 Exhibit a Highly Proliferative and Activated Transcriptomic Signature after Chronic Antigen Stimulation

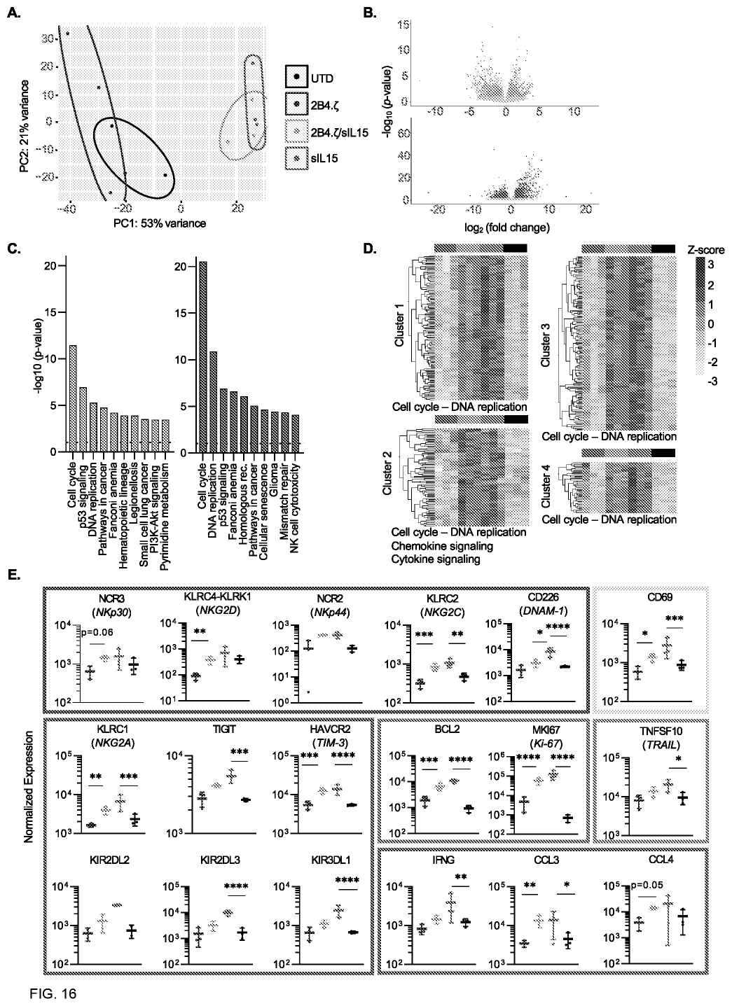

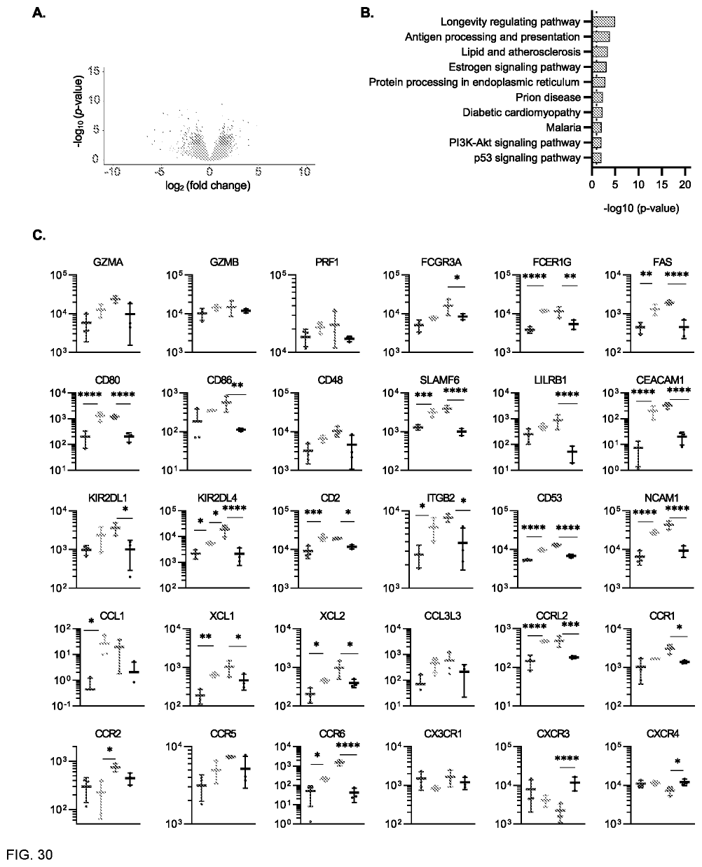

We evaluated the transcriptional programs of our NK cells on the tenth day of continuous antigen stimulation. NK cells were isolated, and RNA libraries prepared and used for RNAseq analysis. Samples clustered by IL15 secretion, with overlap between 2B4.ζ/sIL15 and sIL15 conditions. These clearly separated from NK cells that did not secrete IL15 (UTD and 2B4.ζ; FIG. 16A). Differential gene expression analysis (DESeq2) of 2B4.ζ/sIL15 vs 2B4.ζ and sIL15 vs UTD NKs identified in the IL15-secreting NK cells differences in expression of genes in the pathways of DNA replication, cell cycle progression, and NK cell mediated cytotoxicity (FIG. 16B, FIG. 16C). KEGG enrichment analysis revealed cell cycle progression as the top-ranked pathway for both comparisons (FIG. 16C). No biologically relevant pathways were found to be significantly enriched when comparing IL15-secreting CAR-NK cells to non-CAR sIL15 NKs (FIG. 30A, FIG. 30B). Hierarchical clustering of differentially expressed genes also revealed upregulation of genes involved predominantly in cell cycle progression, chemokine, and cytokine signaling in IL15-secreting NK conditions (FIG. 16D, Table 2 below). We used this dataset to evaluate differential expression of molecules of biological relevance. 2B4.ζ/sIL15 and sIL15 NK cells had higher expression of genes encoding for NK activating receptors (NCR2 (NKp44), NCR3 (NKp30), KLRC2 (NKG2C), KLRC4-KLRK1 (NKG2D), CD226 (DNAM-1), FCGR3A (CD16)), adaptor molecules (FCER1G (FcεR1γ)), death receptor ligands (TNFSF10 (TRAIL)), granzyme (GZMA (granzyme A)), proinflammatory cytokines and chemokines (IFNG (IFN-γ), CCL1, CCL3, CCL4, XCL1, XCL2, CCL3L3), activation markers (CD69), proliferation markers (MKI67 (Ki-67)), anti-apoptosis regulators (BCL2), and adhesion molecules (ITGB2 (integrin-P2), CD2, CD53; FIG. 16E, FIG. 30C). We also observed upregulation of select inhibitory receptors (NKG2A, CEACAM1, LILRB1) including inhibitory Killer Ig-Like Receptors (KIRs; KIR2DL1, KIR2DL2, KIR2DL3, KIR2DL4, KIR3DL1) as well as “checkpoint” molecules (HAVCR2 (TIM-3), TIGIT; FIG. 16E, FIG. 30C). KIR are subject to extensive regulatory splicing and there is an inability to distinguish KIR with identical extracellular domains but functionally disparate intracellular tails by flow cytometry, making transcriptional analysis necessary and complementary for study of KIR expression. Analysis of the chemokine receptor expression showed upregulation of CCRL2, CCR1, CCR5 and CCR6 with similar or lower expression of CX3CR1, CXCR3 and CXCR4 in IL15-secreting NKs (FIG. 30C).

TABLE 2
Genes included in the clusters 1-4 shown in FIG. 16D (listed from
top to bottom of the cluster).

| Cluster 1 | Cluster 2 | Cluster 3 | Cluster 4 |
|---|---|---|---|
| RAD54B | CD9 | DTL | CTNNA1 |
| MSH5 | APOD | CHEK2 | ANKRD35 |
| EHHADH | SMOX | CDC45 | EFCAB11 |
| SIGLEC7 | GPA33 | TROAP | RCCD1 |
| HCG27 | EGR2 | CDKN2C | CDKN2B-ASI |
| B4GALT6 | PAQR4 | CCNE1 | FAM72B |
| ZNF90 | E2F1 | PHF19 | C4orf33 |
| SAPCD1 | ORC1 | MAD2L1 | ANXA4 |
| BLVRA | CDT1 | WDR76 | CD86 |
| MCOLN2 | TONSL | IKZF4 | SIM1 |
| NEDD4 | HIST1H3G | LRP8 | TMPO |
| ACYP1 | POLD1 | LIN9 | FAM72D |
| ACOT9 | HIST1H1B | PTTG1 | FBXO43 |
| SORT1 | HIST1H2BM | KIF23 | CMPK2 |
| CDC42BPA | HAUS8 | CCDC150 | FN1 |
| KIF18A | HIST1H2AL | EPB41L2 | CRTAM |
| SAPCD1 | CENPM | HIST1H1D | KIAA1524 |
| STAP1 | NHS | ENTPD1 | KPNA2 |
| ANO6 | CD84 | PHTF2 | SGOL2 |
| DBF4 | CCNF | RSAD2 | CCDC18 |
| LOC100507002 | TP73 | GEM | ERI2 |
| CENPK | TNC | CASC5 | BRCA2 |
| KDELC2 | TIAM1 | NUF2 | CDH17 |
| GBP4 | PTPRG | UBE2T | MELK |
| GGCT | GOLGA8G | FANCI | CDC7 |
| SIRPG | ADGRL2 | RAD51AP1 | RRM2 |
| BARD1 | ZFHX3 | NUSAP1 | HJURP |
| GZMA | WNT7B | POLQ | DIAPH3 |
| DGKA | DRAXIN | NCAPH | SHCBP1 |
| FBXO44 | GOLGA8DP | CLSPN | KNTC1 |
| CENPQ | BCL2L11 | UBE2C | E2F8 |
| ARL6IP6 | GOLGA8F | STMN1 | KIF20A |
| NEIL3 | FRMD5 | FOXM1 | DEPDC1B |
| ELOVL6 | ACVR1B | SKA1 | TMPO |
| CENPE | CHRM3-AS2 | TYMS | TACC3 |
| CCNE2 | ADAM19 | C2orf48 | DDIAS |
| DPP4 | NCR2 | CDC25C | KIF15 |
| HTATSF1 | VWA2 | SPC25 | TTK |
| FBXO5 | DYSF | GINS2 | E2F7 |
| YEATS4 | RGS16 | LOC81691 | CEP55 |
| SMC4 | FAM221A | MAL | NEK2 |
| STARD4 | CCL3L3 | EME1 | ACAT2 |
| CCNG1 | MIR210HG | BRCA1 | OAS3 |
| KIR2DL4 | CCL4 | XRCC2 | INCENP |
| TNS4 | CCL3 | SGOL1 | |
| MYBL1 | HIST2H2AB | CCDC34 | |
| GPR171 | HAPLN3 | PTGIS | |
| KIF20B | TNFSF12 | PTPN3 | |
| SYCP2 | OMD | ASF1B | |
| DCK | PSMC3IP | PKMYT1 | |
| NTRK2 | HACD1 | MYBL2 | |
| DMRT2 | SDC4 | E2F2 | |
| NEDD9 | DTYMK | RAD54L | |
| CEP152 | LRRC20 | EDA2R | |
| ARHGAP11A | SMCO4 | LOC100506985 | |
| PDGFRB | PRADC1 | RCAN3 | |
| SOCS2 | LTA | HIST1H3B | |
| ARNTL2 | FAAP24 | MCM4 | |
| ERI1 | FANCC | SPAG5 | |
| IFI44 | CENPH | GINS3 | |
| EZH2 | C17orf53 | ORC6 | |
| GBP1 | TK1 | SAPCD2 | |
| OPHN1 | LOC642846 | CKS1B | |
| LOC100288637 | UNQ6494 | TUBA1B | |
| FBXO2 | TNFRSF8 | TUBB | |
| AFAP1L2 | C16orf59 | HIST1H2BH | |
| LTB | TRAIP | RMI2 | |
| PIF1 | FBXO6 | CCNB1 | |
| LAIR2 | FANCG | EXO1 | |
| EPDR1 | DMD | CDC6 | |
| HOXB6 | IFI27L1 | BUB1B | |
| CD80 | AURKB | CENPI | |
| NUGGC | STXBP5L | GTSE1 | |
| RNASE4 | DMC1 | TICRR | |
| CHEK1 | OIP5 | IQGAP3 | |
| CDKN3 | MCOLN3 | DLGAP5 | |
| CREM | APOBEC3B | CIT | |
| KIF11 | KIF4A | AURKA | |
| PLK4 | KIF24 | BCL2 | |
| RBBP8 | TMEM106C | ASPM | |
| HMGB2 | NRM | STIL | |
| DNAJC9 | CENPN | PRIM1 | |
| FAM72C | CENPP | CDK1 | |
| PRTFDC1 | CDK2 | TPX2 | |
| LEF1 | UBASH3B | ANLN | |
| TNFSF8 | | PRR11 | |
| FAS | | CDCA2 | |
| ATAD2 | | PRC1 | |
| CENPU | | MKI67 | |
| RRM1 | | ESCO2 | |
| TCF19 | | TOP2A | |
| BRIP1 | | KIF14 | |
| DEPDC1 | | HMMR | |
| CKAP2L | | CCNB2 | |
| NCAPG | | CDCA3 | |
| ECT2 | | CDCA8 | |
| NDC80 | | KIF18B | |
| ZNF367 | | MCM10 | |
| RGS6 | | CDC20 | |
| TJP1 | | KIFC1 | |
| ITGA2 | | TIMELESS | |
| CEACAM1 | | KIF2C | |
| HOXB7 | | KIAA0101 | |
| MB21D2 | | ZWINT | |
| SLAMF1 | | WDR62 | |
| LOC100294145 | | CDCA5 | |
| FANCD2 | | PLK1 | |
| RNF157 | | RAD51 | |
| C1orf112 | | CDC25A | |
| MASTL | | DSN1 | |
| MBOAT2 | | SUV39H1 | |
| POLE2 | | POC1A | |
| C18orf54 | | CBR3 | |
| LOC399815 | | FDXR | |
| ZNF730 | | LIG1 | |
| TEX30 | | BIRC5 | |
| USP18 | | ESPL1 | |
| HMSD | | UHRF1 | |
| LINC00892 | | CENPW | |
| SERPINE2 | | PCNA | |
| ACTA2 | | CENPA | |
| TSPAN5 | | WDHD1 | |
| B4GALNT1 | | TRIP13 | |
| LINC00504 | | DHFR | |
| NDFIP2 | | FAM111B | |
| NCAM1 | | SKA3 | |
| | | SMC2 | |
| | | CCNA2 | |
| | | GGH | |
| | | STX3 | |
| | | RACGAP1 | |
| | | DSCC1 | |
| | | RBL1 | |
| | | ANXA9 | |
| | | APOL4 | |
| | | C21orf58 | |
| | | GINS4 | |
| | | MCM2 | |
| | | HIRIP3 | |
| | | MCM3 | |
| | | MTFR2 | |
| | | GINS1 | |
| | | NCAPG2 | |
| | | CHAF1B | |
| | | RPL39L | |
| | | NPC2 | |
| | | IFNLR1 | |
| | | OAS1 | |
| | | CKS2 | |
| | | GMNN | |
| | | KDELC1 | |
| | | ARHGAP11B | |

### Constitutive IL15 Expression Improves NK Cell In Vivo Persistence, but May Cause Lethal Toxicity

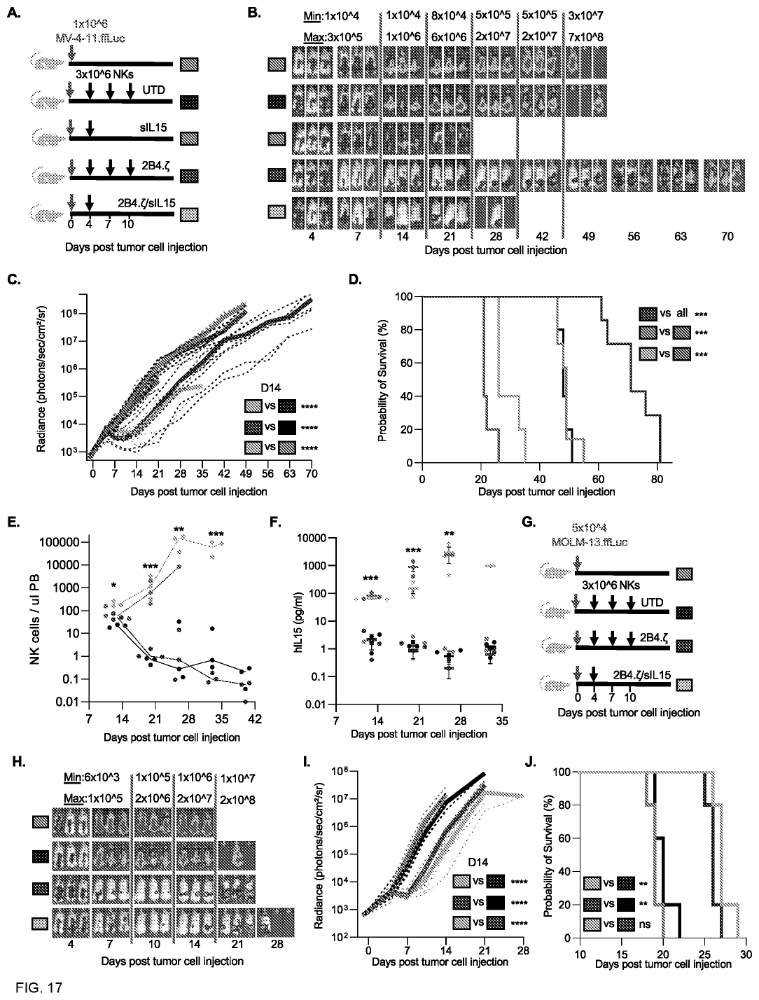

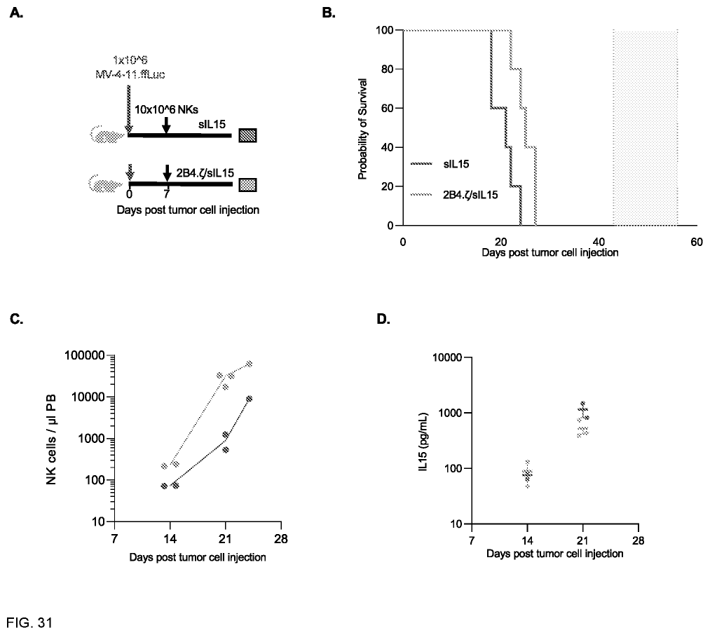

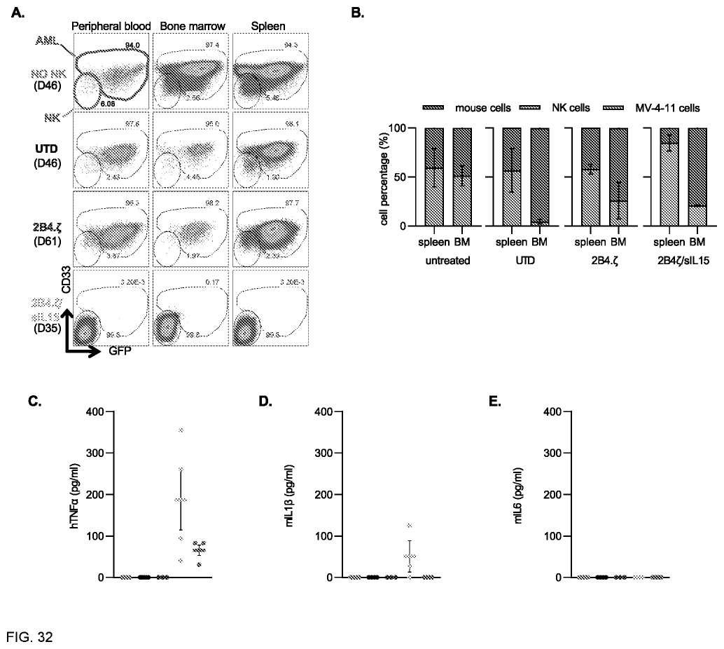

We next evaluated IL15-secreting NK cells in two AML xenograft models. In our first experiment, we used our MV-4-11 xenograft model (FIG. 13A) and our previous NK cell dosing regimen (FIG. 31A). Surprisingly, MV-4-11 engrafted mice treated with 10×10⁶ IL15-secreting NK cells had early mortality (median survival (days); 2B4.ζ/sIL15: 25; sIL15: 21; n=5 mice each group; FIG. 31B). Circulating human IL15 and NK cell counts both increased in the three weeks from NK cell injection to death (FIGS. 31C-D). Therefore, in our next experiment we decreased our treatment dose of infused NK cells in order to evaluate whether we could mitigate the observed treatment associated mortality. We compared a single decreased dose of 2B4.ζ/sIL15 CAR-NK cells with multiple doses of 2B4.ζ CAR-NK cells (FIG. 17A). Infusion of a single dose of 3×10⁶ 2B4.ζ/sIL15 CAR-NK and three doses of 3×10⁶ 2B4.ζ CAR-NK cells both had transient AML control (FIGS. 17B-C). Three doses of 2B4.ζ CAR-NKs prolonged survival, as compared to mice in all other treatment groups (median survival in days; 2B4.ζ vs UTD and untreated; 71 vs 48 and 49, p<0.001; FIG. 17D). In contrast, even at the lower dose, toxicity and premature death of mice treated with IL15-secreting NK cells again occurred (median survival in days); 2B4.ζ/sIL15 vs untreated; 26 vs 49, p<0.001; sIL15 vs untreated; 21 vs 49, p<0.001; FIG. 17D). Analysis of peripheral blood showed in vivo expansion of 2B4.ζ/sIL15 and sIL15 NK cells, with declining NK cell counts again observed in non-IL15 secreting NK cell cohorts (FIG. 17E). Bone marrow and spleen analysis at necropsy of mice treated with 2B4.ζ/sIL15 CAR-NKs revealed high numbers of infiltrating NK cells (FIGS. 32A-B). We identified increasing systemic hIL15 in mice treated with 2B4.ζ/sIL15 and sIL15 NKs (FIG. 17F). In addition, high levels of hTNFα, low levels of mIL1β and negligible levels of mIL6 were detected in the blood of mice treated with 2B4.ζ/sIL15 CAR-NK cells (FIGS. 32C-E).

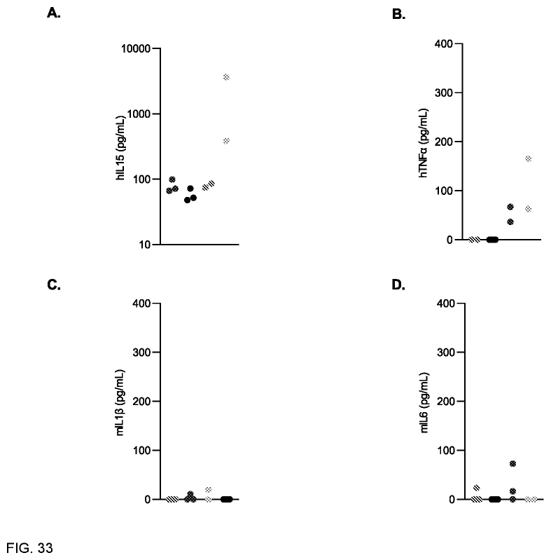

We further evaluated our CAR-NK cells in a more aggressive MOLM-13 xenograft model (FIG. 17G). A single dose of 3×10⁶2B4.ζ/sIL15 CAR-NK cells and three doses of 3×10⁶ 2B4.ζ CAR-NK cells again had equivalent transient antitumor control (FIG. 17H-I). In contrast to the lethal toxicity seen in the MV-4-11 model, both treatment strategies prolonged survival in MOLM-13 engrafted mice (median survival in days; 2B4.ζ vs UTD; 26 vs 20, p<0.01; 2B4.ζ/sIL15 vs UTD; 27 vs: 20, p<0.01; FIG. 17J). Analysis of peripheral blood at necropsy showed circulating systemic hIL15 and hTNFα, but negligible levels of mIL1β and mIL6 (FIGS. 33A-D).

### DISCUSSION

Among other things, we have described the generation and functional evaluation of chimeric receptors targeting the AML-associated antigen CD123 and expressed in primary human NK cells. The molecular domains distal to the extracellular scFv consisted of various combinations of NK-specific activation moieties. We identified a 2B4.ζ CAR as having similar in vitro functionality to the well-studied 4-1BB.ζ CAR,³⁵ with high surface expression, antigen-specific activation, and cytotoxicity. The in vivo anti-tumor effect was more pronounced when AML-engrafted mice were treated with 2B4.ζ CAR-NK cells as compared to those treated with 4-1BB.ζ CAR-NK cells, which suggests a potential additive effect of 2B4 and CD3ζ signaling. However, this in vivo effect was short-lived and was accompanied by circulating NK-cell decline. We thus engineered our 2B4.ζ CAR-NK cells with an IL15 transgene to promote IL15-mediated activation, proliferation, and survival. Secretion of IL15 stimulated CAR-NK cell expansion both in vitro and in vivo, and enhanced short- and long-term anti-AML cytotoxicity. This bolstered activation profile was confirmed by immunophenotypic and transcriptomic analysis in the setting of chronic stimulation.

However, in one in vivo AML model, constitutive IL15 secretion caused dramatic NK-cell expansion, high levels of circulating human pro-inflammatory cytokines, and was associated with early death. Treatment with three doses of non-IL15-secreting CAR-NK cells prolonged survival without systemic toxicity, but anti-tumor efficacy was transient.

CARs are used to enhance and redirect immune effector cells against cancer cells. CAR-T cells have been extensively investigated in preclinical and clinical models of AML.⁹ However, there are limited preclinical animal studies^{36,37} and only 3 active clinical trials (NCT04623944, NCT05008575, NCT02742727) testing CAR-NK cells as AML therapy. There is a single completed anti-AML CAR-NK cell trial with evaluable data (NCT02944162). This study tested an engineered anti-CD33 CAR-NK92 cell line in 3 patients with relapsed and refractory AML. The CD33-CAR NK92 cell infusion was safe, but the treatment had minimal antitumor efficacy.³⁸ We aimed to enhance the molecular functionality of our CAR by using NK cell-specific receptor domains. We identified the 2B4TM-2B4-CD3ζ CAR as optimal for NK cell expression and activation. The end-result of 2B4 downstream signaling includes synergy with other NK activating receptors.^{17,39} Protein interactions including 2B4 occur inside membrane (“lipid”) rafts. We chose to use the 2B4 transmembrane domain to localize the CAR within lipid rafts,⁴⁰ and found that CARs containing a 2B4 transmembrane domain had high surface density and stable CAR expression. Tandem CD3ζ was also important, highlighted by the observed functional differences between 2B4.ζ and 2B4 CAR-NK cells.

To our knowledge, we describe the first study investigating both the in vitro and in vivo anti-AML functionality of peripheral blood derived CAR-NK cells (PB-NKs) targeting CD123. We chose the peripheral blood (PB) of healthy donors as the source of our CAR-NK cells. PB-derived NK cells (PB-NKs) can be isolated through apheresis and expanded in large scale using feeder cells.²⁶ PB-NKs are functionally mature, with high activating receptor expression and cytotoxic potency. PB-NKs also display higher levels of Killer Ig-Like Receptors (KIRs) compared to other NK cell products derived from alternative sources. KIR expression is indicative of more complete NK cell licensing.⁴¹ Donor-derived, allogeneic PB-NK cells have therapeutic potential due to their relative safety, immediate availability once manufactured and stored, and reduced manufacturing costs as compared to per-patient manufacture of autologous cell therapy products.^{12,13} Historically, one challenge facing PB-NK cell engineering was that of poor viral and non-viral genetic modification.⁴² With our method, we are able to achieve high levels of PB-NK transduction. All of our CARs were stably expressed on the surface of primary NK cells, though inter-CAR variability in surface density was observed.

NK cells have a natural lifespan of approximately 2 weeks in humans. The success of adoptively transferred cellular therapies for cancer is determined, in part, by effector cell persistence. The use of systemic cytokine supplementation is one common strategy employed to support prolongation of NK cell survival. The short half-life of infused IL15 necessitates frequent or continuous administration, and systemic toxicity is common.⁴³⁻⁴⁶ IL15 super agonists, like N-803 (formerly known as ALT-803), have a longer half-life and can mimic physiological IL15 trans presentation, but administration can cause inflammatory toxicities.⁴⁷ The administration of N-803 has been effective at promoting NK cell proliferation and anti-tumor efficacy against hematologic malignancies, with expected associated fever, chills, and injection site rashes observed.⁴⁸⁻⁵¹ Another strategy that has been successful in specifically supporting in vivo CAR-NK cell survival is engineering constitutive activating cytokine expression.⁵² This approach has been shown to be safe in a clinical trial using CD19-CAR NK cells against CD19+ lymphoid malignancies.⁵³ The demonstrated safety profile motivated us to also test transgenic IL15 expression with a goal of enhanced in vivo CAR-NK cell persistence. We found NK cells subject to activation with constantly available IL15 exhibited enhanced and sustained in vitro and in vivo functionality. Though we concluded this to be resultant from specific activation above baseline, it is possible that our NK cells had been rendered “cytokine addicted”⁵⁴ due to ex vivo culture conditions that include supplemental IL2. In this case, the cohorts of cells without engineered IL15 secretion may have become dysfunctional once removed from a state of trophic cytokine availability.

Treatment with IL15 secreting CAR-NK cells caused early death in mice engrafted with MV-4-11 AML. A likely cause of the observed systemic toxicity is severe inflammation due to the dramatic NK cell proliferation associated with high levels of circulating IL15 and other proinflammatory cytokines. A CRS-like syndrome triggered by murine monocytes or other immune cells is unlikely due to our inability to detect common murine proinflammatory cytokines. IL15 stimulation of accelerated leukemic growth was not observed. Clinical signs associated with hyperinflammation (such as weight loss and hunching) are also seen in GVHD and were observed in our premorbid mice. NK cells have the potential to exacerbate subclinical T cell-mediated acute GVHD.¹⁹ We believe that classically defined GVHD is a less likely cause of our observed toxicity due to the absence of human or murine T cells in our NSG model. However, the possibility of lethal toxicity due to NK cell alloreactivity against mouse cells cannot be excluded.

We observed that IL15-secreting CAR-NK cells both prolonged and shortened survival in two xenograft models of AML. Notably, the median survival of mice treated with the same dose of 2B4.ζ/sIL15 NK cells was similar in each model (MOLM-13: 27 days; MV-4-11: 26 days). This could potentially be explained by the aggressiveness of the MOLM-13 model, as these mice died of widespread disease at roughly the same time as we recorded the onset of treatment associated mortality. Our observation of unexpected toxicity begs for expanded study of novel ways to stimulate CAR-NK cell cytokine receptor pathways while circumventing systemic cytokine delivery. Localization of IL15 with membrane tethering⁵⁵ or targeted delivery through the use of oncolytic viruses⁵⁶ are alternative therapeutic strategies. Controllable cytokine expression using engineered inducible systems and safety switches also holds promise. Specific activation of intrinsic gamma-cytokine receptor signaling without the use of a pharmacologic agent is another strategy that has the potential to sustain NK cell function with an improved safety profile.

### Example 3: CD3ζ ITAM Modification does not Alter the Biological Behavior of 2B4.ζ/sIL15 CAR-NKs

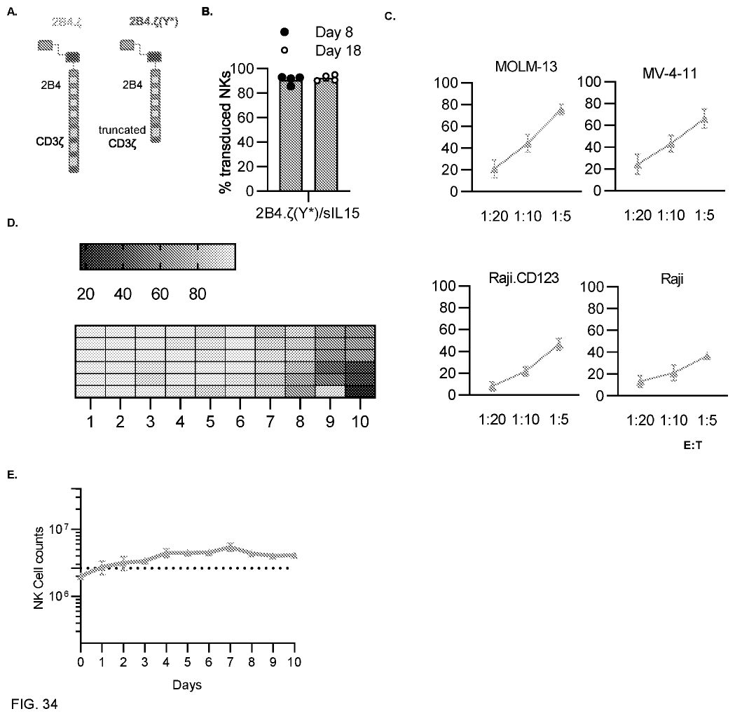

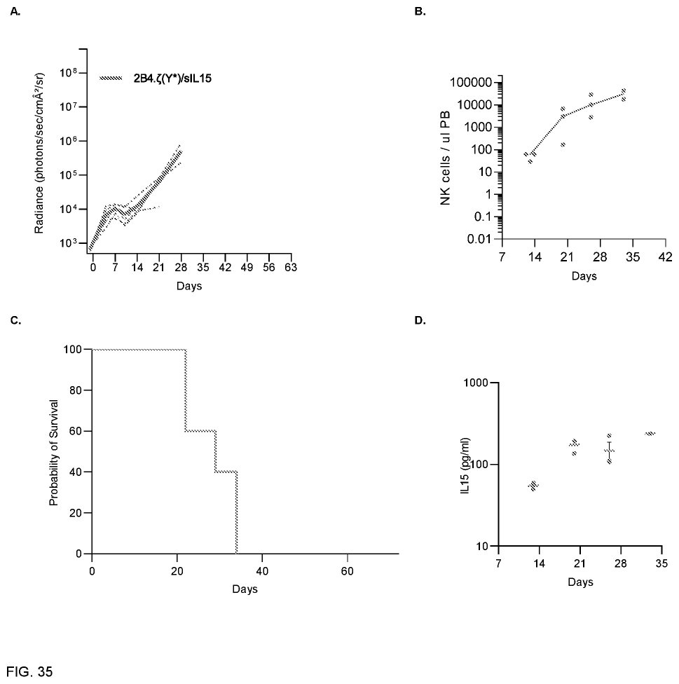

We investigated whether the activated phenotype, anti-AML cytotoxicity, and in vivo functionality of 2B4.ζ/sIL15 CAR-NKs could be changed by CAR (chain molecular mutation. To this end, we truncated the (sequence distal to the proximal intracellular tyrosine activation motif (ITAM).(FIG. 34A) 2B4.ζ/sIL15 and 2B4.ζ(Δ)/sIL15 CAR-NKs were similarly transduced (range CAR (+) cells; ζ: 81.8-96%; ζ(Δ): 85.9-95%; FIG. 14C, 34B). ITAM deletion did not affect cytotoxicity when evaluated in short term (FIG. 14E-G, 34C) and long term serial stimulation (FIG. 15E, 34D) co-culture assays. Similarly, NK cell proliferative capacity was not impacted by this ITAM truncation. (FIG. 15D, 34E) 2B4.ζ/sIL15 and 2B4.ζ(Δ)/sIL15 CAR-NKs displayed similar in vivo behavior in human AML (MV-4-11) engrafted mice (FIG. 17C,E, 35A,B). Lethal toxicity was associated with high systemic levels of circulating human IL-15 (FIG. 17D,F, 35C,D).

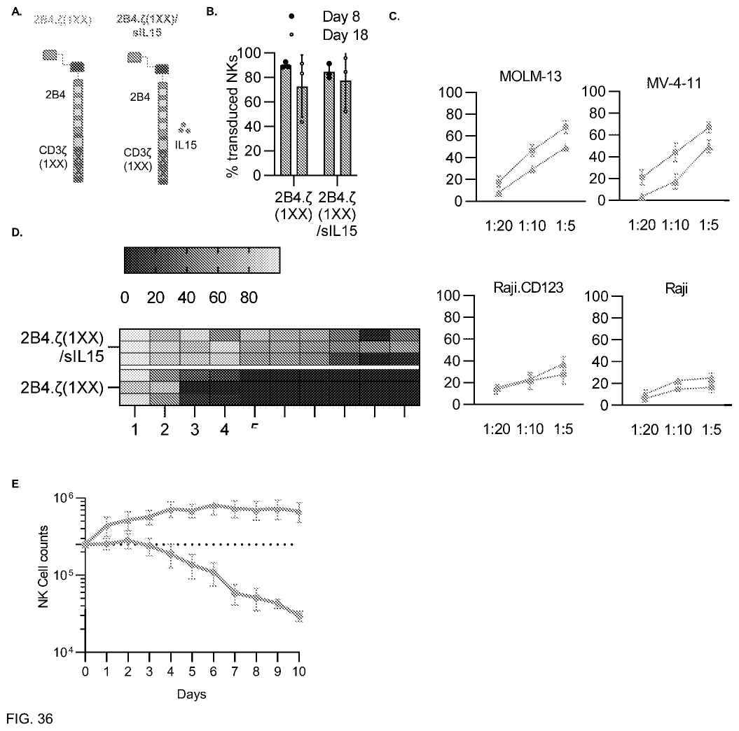

We also tested the mutational inactivation of the distal ITAMs via substitution of the relevant tyrosine with phenylalanine. By preventing tyrosine phosphorylation and subsequent ZAP70 binding, signaling downstream of ITAM activation is abrogated. CARs in both 2B4.ζ(1XX) and 2B4.ζ(1XX)/sIL15 conditions were expressed with high efficiency (FIG. 36A). As seen without ITAM mutation, 2B4.ζ(1XX)/sIL15 CAR-NKs have enhanced anti-AML cytotoxicity as compared to 2B4.ζ(1XX) CAR-NKs, with more potent short-term killing of CD123(+) targets (FIG. 36B) and sustained cytotoxicity and NK cell expansion in when chronically exposed to AML (FIG. 36C-D).

### REFERENCES

1. Gorman, M. F., et al. Outcome for children treated for relapsed or refractory acute myelogenous leukemia (rAML): a Therapeutic Advances in Childhood Leukemia (TACL) Consortium study. Pediatr Blood Cancer 55, 421-429 (2010).
2. Dohner, H., et al. Diagnosis and management of AML in adults: 2017 ELN recommendations from an international expert panel. Blood 129, 424-447 (2017).
3. Yanagisawa, B., et al. Expression of putative leukemia stem cell targets in genetically-defined acute myeloid leukemia subtypes. Leuk Res 99, 106477 (2020).
4. Jordan, C. T., et al. The interleukin-3 receptor alpha chain is a unique marker for human acute myelogenous leukemia stem cells. Leukemia 14, 1777-1784 (2000).
5. Frankel, A., Liu, J. S., Rizzieri, D. & Hogge, D. Phase I clinical study of diphtheria toxin-interleukin 3 fusion protein in patients with acute myeloid leukemia and myelodysplasia. Leuk Lymphoma 49, 543-553 (2008).
6. He, S. Z., et al. A Phase 1 study of the safety, pharmacokinetics and anti-leukemic activity of the anti-CD123 monoclonal antibody CSL360 in relapsed, refractory or high-risk acute myeloid leukemia. Leuk Lymphoma 56, 1406-1415 (2015).
7. Uy, G. L., et al. Flotetuzumab as salvage immunotherapy for refractory acute myeloid leukemia. Blood 137, 751-762 (2021).
8. Greenbaum, U., Mahadeo, K. M., Kebriaei, P., Shpall, E. J. & Saini, N.Y. Chimeric Antigen Receptor T-Cells in B-Acute Lymphoblastic Leukemia: State of the Art and Future Directions. Front Oncol 10, 1594 (2020).
9. Fiorenza, S. & Turtle, C. J. CAR-T Cell Therapy for Acute Myeloid Leukemia: Preclinical Rationale, Current Clinical Progress, and Barriers to Success. BioDrugs (2021).
10. Lee, D. W., et al. ASTCT Consensus Grading for Cytokine Release Syndrome and Neurologic Toxicity Associated with Immune Effector Cells. Biol Blood Marrow Transplant 25, 625-638 (2019).
11. Hines, M. R., et al. Hemophagocytic lymphohistiocytosis-like toxicity (carHLH) after CD19-specific CAR T-cell therapy. Br J Haematol 194, 701-707 (2021).
12. Kohl, U., Arsenieva, S., Holzinger, A. & Abken, H. CAR T Cells in Trials: Recent Achievements and Challenges that Remain in the Production of Modified T Cells for Clinical Applications. Hum Gene Ther 29, 559-568 (2018).
13. Papathanasiou, M. M., et al. Autologous CAR T-cell therapies supply chain: challenges and opportunities? Cancer Gene Ther 27, 799-809 (2020).
14. Das, R. K., Vernau, L., Grupp, S. A. & Barrett, D. M. Naive T-cell Deficits at Diagnosis and after Chemotherapy Impair Cell Therapy Potential in Pediatric Cancers. Cancer Discov 9, 492-499 (2019).
15. Ferrara, J. L., Levine, J. E., Reddy, P. & Holler, E. Graft-versus-host disease. Lancet 373, 1550-1561 (2009).
16. Chester, C., Fritsch, K. & Kohrt, H. E. Natural Killer Cell Immunomodulation: Targeting Activating, Inhibitory, and Co-stimulatory Receptor Signaling for Cancer Immunotherapy. Front Immunol 6, 601 (2015).
17. Lanier, L. L. Up on the tightrope: natural killer cell activation and inhibition. Nat Immunol 9, 495-502 (2008).
18. Verheyden, S. & Demanet, C. NK cell receptors and their ligands in leukemia. Leukemia 22, 249-257 (2008).
19. Shah, N. N., et al. Acute GVHD in patients receiving IL-15/4-1BBL activated NK cells following T-cell-depleted stem cell transplantation. Blood 125, 784-792 (2015).
20. Bachiller, M., Battram, A. M., Perez-Amill, L. & Martin-Antonio, B. Natural Killer Cells in Immunotherapy: Are We Nearly There? Cancers (Basel) 12(2020).
21. Nguyen, R., et al. A phase II clinical trial of adoptive transfer of haploidentical natural killer cells for consolidation therapy of pediatric acute myeloid leukemia. J Immunother Cancer 7, 81 (2019).
22. Shaffer, B. C., et al. Phase II Study of Haploidentical Natural Killer Cell Infusion for Treatment of Relapsed or Persistent Myeloid Malignancies Following Allogeneic Hematopoietic Cell Transplantation. Biol Blood Marrow Transplant 22, 705-709 (2016).
23. Choi, I., et al. Donor-Derived Natural Killer Cell Infusion after Human Leukocyte Antigen-Haploidentical Hematopoietic Cell Transplantation in Patients with Refractory Acute Leukemia. Biol Blood Marrow Transplant 22, 2065-2076 (2016).
24. Lee, D. A., et al. Haploidentical Natural Killer Cells Infused before Allogeneic Stem Cell Transplantation for Myeloid Malignancies: A Phase I Trial. Biol Blood Marrow Transplant 22, 1290-1298 (2016).
25. Du, X., Ho, M. & Pastan, I. New immunotoxins targeting CD123, a stem cell antigen on acute myeloid leukemia cells. J Immunother 30, 607-613 (2007).
26. Fujisaki, H., et al. Expansion of highly cytotoxic human natural killer cells for cancer cell therapy. Cancer Res 69, 4010-4017 (2009).
27. Nowicka, M., et al. CyTOF workflow: differential discovery in high-throughput high-dimensional cytometry datasets. F1000Res 6, 748 (2017).
28. Becht, E., et al. Dimensionality reduction for visualizing single-cell data using UMAP. Nat Biotechnol (2018).
29. Van Gassen, S., et al. FlowSOM: Using self-organizing maps for visualization and interpretation of cytometry data. Cytometry A 87, 636-645 (2015).
30. Love, M. I., Huber, W. & Anders, S. Moderated estimation of fold change and dispersion for RNA-seq data with DESeq2. Genome Biol 15, 550 (2014).
31. Krawczyk, E., Zolov, S. N., Huang, K. & Bonifant, C. L. T-cell Activity against AML Improved by Dual-Targeted T Cells Stimulated through T-cell and IL7 Receptors. Cancer Immunol Res 7, 683-692 (2019).
32. Bonifant, C. L., et al. CD123-Engager T Cells as a Novel Immunotherapeutic for Acute Myeloid Leukemia. Mol Ther 24, 1615-1626 (2016).
33. Zolov, S. N., Rietberg, S. P. & Bonifant, C. L. Programmed cell death protein 1 activation preferentially inhibits CD28.CAR-T cells. Cytotherapy 20, 1259-1266 (2018).
34. Imai, C., et al. Chimeric receptors with 4-1BB signaling capacity provoke potent cytotoxicity against acute lymphoblastic leukemia. Leukemia 18, 676-684 (2004).
35. Imai, C., Iwamoto, S. & Campana, D. Genetic modification of primary natural killer cells overcomes inhibitory signals and induces specific killing of leukemic cells. Blood 106, 376-383 (2005).
36. Gurney, M., et al. CD38 knockout natural killer cells expressing an affinity optimized CD38 chimeric antigen receptor successfully target acute myeloid leukemia with reduced effector cell fratricide. Haematologica Online ahead of print (2020).
37. Salman, H., et al. Preclinical Targeting of Human Acute Myeloid Leukemia Using CD4-specific Chimeric Antigen Receptor (CAR) T Cells and NK Cells. J Cancer 10, 4408-4419 (2019).
38. Tang, X., et al. First-in-man clinical trial of CAR NK-92 cells: safety test of CD33-CAR NK-92 cells in patients with relapsed and refractory acute myeloid leukemia. Am J Cancer Res 8, 1083-1089 (2018).
39. Tangye, S. G., Cherwinski, H., Lanier, L. L. & Phillips, J. H. 2B4-mediated activation of human natural killer cells. Mol Immunol 37, 493-501 (2000).
40. Gutgemann, S. A., Sandusky, M. M., Wingert, S., Claus, M. & Watzl, C. Recruitment of activating NK-cell receptors 2B4 and NKG2D to membrane microdomains in mammalian cells is dependent on their transmembrane regions. Eur J Immunol 45, 1258-1269 (2015).
41. Kim, S., et al. Licensing of natural killer cells by host major histocompatibility complex class I molecules. Nature 436, 709-713 (2005).
42. Boissel, L., et al. Comparison of mRNA and lentiviral based transfection of natural killer cells with chimeric antigen receptors recognizing lymphoid antigens. Leuk Lymphoma 53, 958-965 (2012).
43. Conlon, K. C., et al. Redistribution, hyperproliferation, activation of natural killer cells and CD8 T cells, and cytokine production during first-in-human clinical trial of recombinant human interleukin-15 in patients with cancer. J Clin Oncol 33, 74-82 (2015).
44. Conlon, K. C., et al. IL15 by Continuous Intravenous Infusion to Adult Patients with Solid Tumors in a Phase I Trial Induced Dramatic NK-Cell Subset Expansion. Clin Cancer Res 25, 4945-4954 (2019).
45. Cooley, S., et al. First-in-human trial of rhIL-15 and haploidentical natural killer cell therapy for advanced acute myeloid leukemia. Blood Adv 3, 1970-1980 (2019).
46. Miller, J. S., et al. A First-in-Human Phase I Study of Subcutaneous Outpatient Recombinant Human IL15 (rhIL15) in Adults with Advanced Solid Tumors. Clin Cancer Res 24, 1525-1535 (2018).
47. Knudson, K. M., Hodge, J. W., Schlom, J. & Gameiro, S.R. Rationale for IL-15 superagonists in cancer immunotherapy. Expert Opinion on Biological Therapy 20, 705-709 (2020).
48. Romee, R., et al. First-in-human phase 1 clinical study of the IL-15 superagonist complex ALT-803 to treat relapse after transplantation. Blood 131, 2515-2527 (2018).
49. Rosario, M., et al. The IL-15-Based ALT-803 Complex Enhances FcγRIIIa-Triggered NK Cell Responses and <em>In Vivo</em>Clearance of B Cell Lymphomas. Clinical Cancer Research 22, 596-608 (2016).
50. Xu, W., et al. Efficacy and Mechanism-of-Action of a Novel Superagonist Interleukin-15: Interleukin-15 Receptor αSu/Fc Fusion Complex in Syngeneic Murine Models of Multiple Myeloma. Cancer Research 73, 3075-3086 (2013).
51. Foltz, J. A., et al. Phase I Trial of N-803, an IL15 Receptor Agonist, with Rituximab in Patients with Indolent Non-Hodgkin Lymphoma. Clinical Cancer Research 27, 3339-3350 (2021).
52. Liu, E., et al. Cord blood NK cells engineered to express IL-15 and a CD19-targeted CAR show long-term persistence and potent antitumor activity. Leukemia 32, 520-531 (2018).
53. Liu, E., et al. Use of CAR-Transduced Natural Killer Cells in CD19-Positive Lymphoid Tumors. N Engl J Med 382, 545-553 (2020).
54. Duke, R. C. & Cohen, J. J. IL-2 addiction: withdrawal of growth factor activates a suicide program in dependent T cells. Lymphokine Res 5, 289-299 (1986).
55. Imamura, M., et al. Autonomous growth and increased cytotoxicity of natural killer cells expressing membrane-bound interleukin-15. Blood 124, 1081-1088 (2014).
56. Ma, R., et al. An Oncolytic Virus Expressing IL15/IL15Ralpha Combined with Off-the-Shelf EGFR-CAR NK Cells Targets Glioblastoma. Cancer Res 81, 3635-3648 (2021).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. An engineered cell comprising: a chimeric antigen receptor (CAR) polypeptide comprising a hinge domain, a transmembrane domain, and/or an intracellular domain.

2. The engineered cell of claim 1, wherein the cell comprises an immune cell, wherein the immune cell comprises natural killer (NK) cells or T cells.

3. The engineered cell of claim 2, wherein the immune cell comprises NK cells.

4. The engineered cell of claim 1, further comprising a signaling domain, an activating domain, a stimulatory domain, an antigen recognition domain, or a co-stimulatory domain.

5. The engineered cell of claim 1, wherein the chimeric antigen receptor (CAR) polypeptide comprises a signaling domain that comprises an immunoreceptor tyrosine-based activation motif (ITAM).

6. The engineered cell of claim 1, wherein the hinge domain comprises 2B4 comprising the sequence QDCQNAHQEFRFWP (SEQ ID NO: 1), FcεR1γ comprising the sequence LGEPQ (SEQ ID NO: 2), or DAP10 comprising the sequence QTTPGERSSLPAFYPGTSGSCSGCGSLSLP (SEQ ID NO: 3).

7. The engineered cell of claim 1, wherein the transmembrane region comprises 2B4, comprising the sequence FLVIIVILSALFLGTLACFCV (SEQ ID NO: 29), FcεR1γ comprising the sequence LCYILDAILFLYGIVLTLLYC (SEQ ID NO: 30), or DAP10 comprising the sequence LLAGLVAADAVASLLIVGAVF (SEQ ID NO: 31).

8. The engineered cell of claim 1, wherein the intracellular domain comprises 2B4 (SEQ ID NO: 4), CD3ζ (1XX) (SEQ ID NO: 5), CD3ζ (SEQ ID NO: 6), CD3ζ (1Δ), (SEQ ID NO: 7) FcεR1γ (SEQ ID NO: 8), or DAP10 (SEQ ID NO: 9).

9. The engineered cell of claim 1, wherein the cell further comprises an internal ribosome entry site (IRES) domain.

10. The engineered cell of claim 1, wherein the cell further comprises a cytokine.

11-13. (canceled)

14. The engineered cell of claim 1, wherein the CAR comprises the structure: 2B4.z(truncated).IRES.secrIL15, 2B4.z(1XX).IRES.secIL15—2B4.z.IRES.secIL15.

15. A pharmaceutical composition that comprises one or more engineered cells of claim 1.

16. A kit that comprises 1) engineered cells of claim 1 and 2) instructions for use of the cells to treat cancer.

17. A method of treating a subject suffering from cancer, comprising administering to the subject the engineered cells or composition of claim 1.

18. A method of treating a subject suffering from acute myeloid leukemia (AML), comprising administering to the subject the engineered cells or composition of claim 1.

19. An isolated natural killer (NK) cell comprising a chimeric antigen receptor (CAR) polypeptide comprising an antigen specific binding domain, a hinge domain, a transmembrane domain, an intracellular domain, a co-stimulatory receptor or combinations thereof, wherein the antigen specific binding domain specifically binds to CD123.

20-32. (canceled)

33. A chimeric antigen receptor (CAR) comprising an antigen specific binding domain, a hinge domain, a transmembrane domain, and/or an intracellular domain.

34. The chimeric antigen receptor of claim 33, further comprising a signaling domain, an activating domain, a stimulatory domain, a co-stimulatory domain or combinations thereof.

35. (canceled)

36. The chimeric antigen receptor of claim 33, wherein the hinge domain comprises 2B4 comprising the sequence QDCQNAHQEFRFWP (SEQ ID NO: 1), FcεR1γ comprising the sequence LGEPQ (SEQ ID NO: 2), or DAP10 comprising the sequence QTTPGERSSLPAFYPGTSGSCSGCGSLSLP (SEQ ID NO: 3).

37-53. (canceled)

54. The chimeric antigen receptor of claim 33 wherein the CAR comprises an antigen specific binding domain, a hinge domain, a transmembrane domain, an intracellular domain, a co-stimulatory receptor or combinations thereof, wherein the antigen specific binding domain specifically binds to CD123.
